(19) United States
(12) Patent Application Publication
(10) Pub. No.: US 2024/0325321 A1
(43) Pub. Date: Oct. 3, 2024

(54) COMPOSITIONS AND METHODS FOR INHIBITING PLpro PROTEASE ACTIVITY AND FOR PREVENTING AND TREATING SARS-CoV-2 INFECTION

(71) Applicant: Arizona Board of Regents on Behalf of the University of Arizona, Tucson, AZ (US)

(72) Inventors: Jun Wang, Tucson, AZ (US); Yanmei Hu, Tucson, AZ (US); Chunlong Ma, Tucson, AZ (US); Naoya Kitamura, Tucson, AZ (US)

(21) Appl. No.: 18/281,737

(22) PCT Filed: Mar. 11, 2022
(86) PCT No.: PCT/US2022/019942

§ 371 Date: Sep. 12, 2023

(51) Int. Cl. A61K 31/137; A61K 31/166; A61K 31/18; A61K 31/40; A61K 31/4045; A61K 31/415; A61K 31/416; A61K 31/417; A61K 31/4192; A61K 31/437; A61K 31/4406; A61K 31/4412; A61K 31/47; A61K 31/472; A61K 31/4965; A61K 31/513; A61K 31/538; A61K 31/69; A61P 31/14

(52) U.S. Cl. CPC A61K 31/137; A61K 31/166; A61K 31/18; A61K 31/40; A61K 31/4045; A61K 31/415; A61K 31/416; A61K 31/417; A61K 31/4192; A61K 31/437; A61K 31/4406; A61K 31/4412; A61K 31/47; A61K 31/472; A61K 31/4965; A61K 31/513; A61K 31/538; A61K 31/69; A61P 31/14

(57) ABSTRACT

This invention is in the field of medicinal chemistry and relates to a new class of small-molecules having a N-methyl-1-(naphthalen-1-yl)ethan-1-amine (or similar) structure (e.g.: Formula I) which function as inhibitors of the SARS-CoV-2 papain-like protease (PL^{pro}), which function as therapeutics for the treatment of viral infection characterized with PL^{pro} protease activity and/or expression (e.g., COVID-19), and which function as therapeutics for the treatment of other conditions characterized with PL^{pro} protease activity and/or expression.

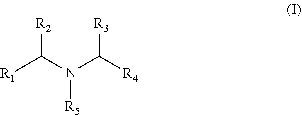

### CROSS REFERENCE TO RELATED APPLICATION

This application claims priority to and the benefit of U.S. Provisional Application No. 63/160,086, filed Mar. 12, 2021, which is hereby incorporated by reference in its entirety.

### FIELD OF THE INVENTION

This invention is in the field of medicinal chemistry and relates to a new class of small-molecules having a N-methyl-1-(naphthalen-1-yl)ethan-1-amine (or similar) structure (e.g.,

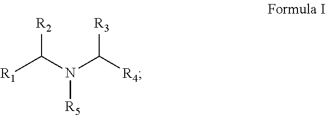

) which function as inhibitors of the SARS-CoV-2 papain-like protease (PL^{pro}), which function as therapeutics for the treatment of viral infection characterized with PL^{pro} protease activity and/or expression (e.g., COVID-19), and which function as therapeutics for the treatment of other conditions characterized with PL^{pro} protease activity and/or expression.

### INTRODUCTION

The COVID-19 pandemic has a significant impact on global economy and public health, and there is an urgent need of therapeutic interventions. Encouraging progress has been made in developing mRNA vaccines including the Pfizer BNT162b2 and Moderna mRNA-1273. For small molecule antivirals, the viral polymerase inhibitor remdesivir gained FDA approval on Oct. 22, 2020. Among the other drug targets that are being explored at different stages of preclinical and clinical development, the viral main protease (M^{pro}), also called 3-chymotrypsin-like protease (3CL^{pro}), is one of the high profile antiviral drug targets.

Research on other aspects of COVID-19, including epidemiology and genome sequencing, has provided useful insights into the new virus. The complete genome sequencing showed that the virus belonged to a large family of coronaviruses and is closely related to Severe Acute Respiratory Syndrome-Coronavirus (SARS-CoV). It encodes for two large polyproteins that are further processed by virally encoded cysteine proteases, namely, the papain-like protease (PL^{pro}) and the 3-chymotrypsin-like protease (3CL^{pro}, also known as the main protease—Mpro). The processing of the viral polyproteins is essential for maturation and infectivity of the virus. Because of the crucial roles these proteases play in the viral life-cycle, they are important targets for antiviral drug design.

Improved pharmaceutical agents capable of inhibiting PL^{pro} protease activity are desperately needed. Improved therapies for treating COVID-19 and conditions characterized with PL^{pro} protease activity are desperately needed.

The present invention addresses these needs.

### SUMMARY

SARS-CoV-2 encodes two cysteine proteases, the main protease (M^{pro}) and the papain-like protease (PL^{pro}). Both proteases are validated antiviral drug targets and play essential roles in viral replication. It has been shown that M^{pro} cleaves the viral polyprotein at more than 11 sites, while PL^{pro} cleaves the viral polyprotein at 3 sites.

Experiments conducted during the course of developing embodiments for the present invention developed a FRET-based enzymatic assay for the PL^{pro}, which was used in a high-throughput screening to identified potent PL^{pro} inhibitors. Several novel compounds were identified. Structure-activity relationship studies were performed to further optimize the potency and selectivity index of these hits. This effort led to a number of potent SARS-CoV-2 PL^{pro} inhibitors with a new composition of matter having a N-methyl-1-(naphthalen-1-yl)ethan-1-amine (or similar) structure (e.g., compounds Jun9-53-2, Jun9-44-3, Jun9-87-1, Jun9-87-2, Jun9-87-3, and Jun10-69-2 represent highly potent SARS-CoV-2 PL^{pro} inhibitors).

Accordingly, the present invention relates to a new class of small-molecules having a N-methyl-1-(naphthalen-1-yl)ethan-1-amine (or similar) structure which function as inhibitors of the SARS-CoV-2 papain-like protease (PL^{pro}), which function as therapeutics for the treatment of viral infection characterized with PL^{pro} protease activity and/or expression (e.g., COVID-19), and which function as therapeutics for the treatment of other conditions characterized with PL^{pro} protease activity and/or expression.

Certain N-methyl-1-(naphthalen-1-yl)ethan-1-amine (or similar) compounds of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, compounds encompassed within the following formulas are provided:

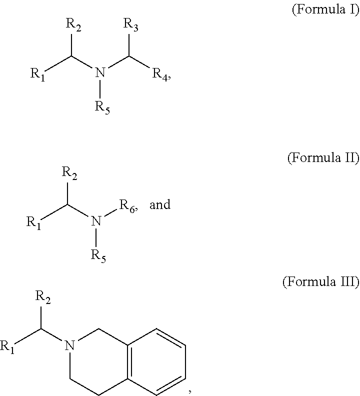

including pharmaceutically acceptable salts, solvates, and/or prodrugs thereof.

Formula I is not limited to a particular chemical moiety for R1, R2, R3, R4, R5, and R6. In some embodiments, the particular chemical moiety for R1, R2, R3, R4, R5, and R6 independently include any chemical moiety that permits the resulting compound to inhibit PL^{pro} protease activity. In some embodiments, the particular chemical moiety R1, R2, R3, R4, R5, and R6 independently include any chemical moiety that permits the resulting compound to prevent viral infection (e.g., COVID-19 infection).

Such embodiments are not limited to a particular definition for R1.

In some embodiments, R1 is selected from hydrogen, methyl,

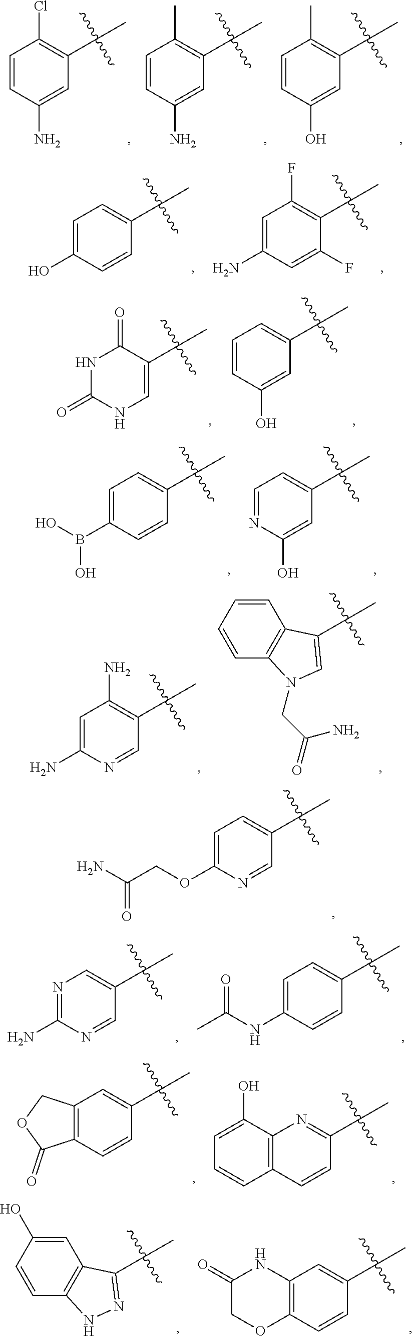

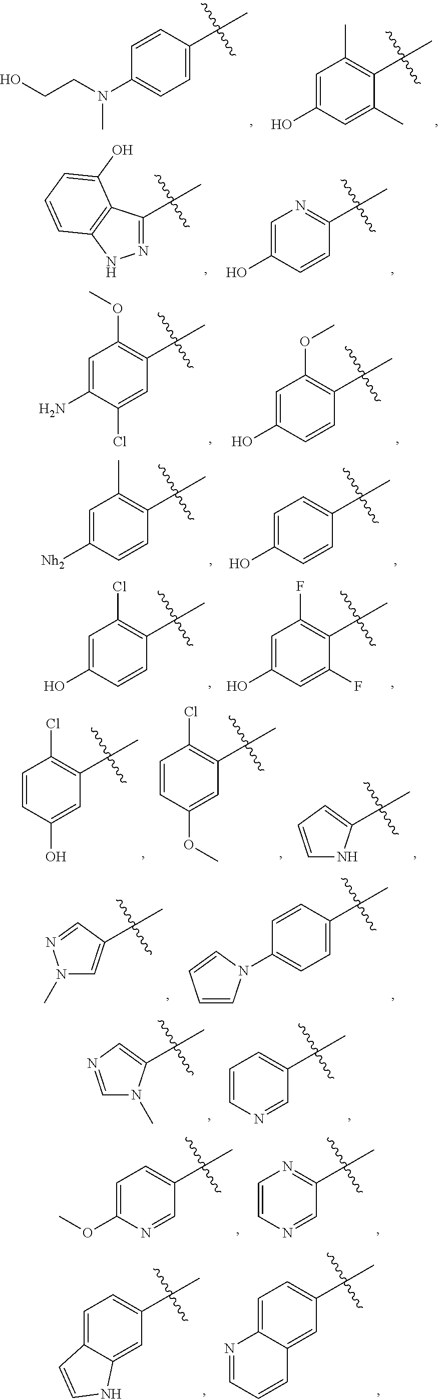

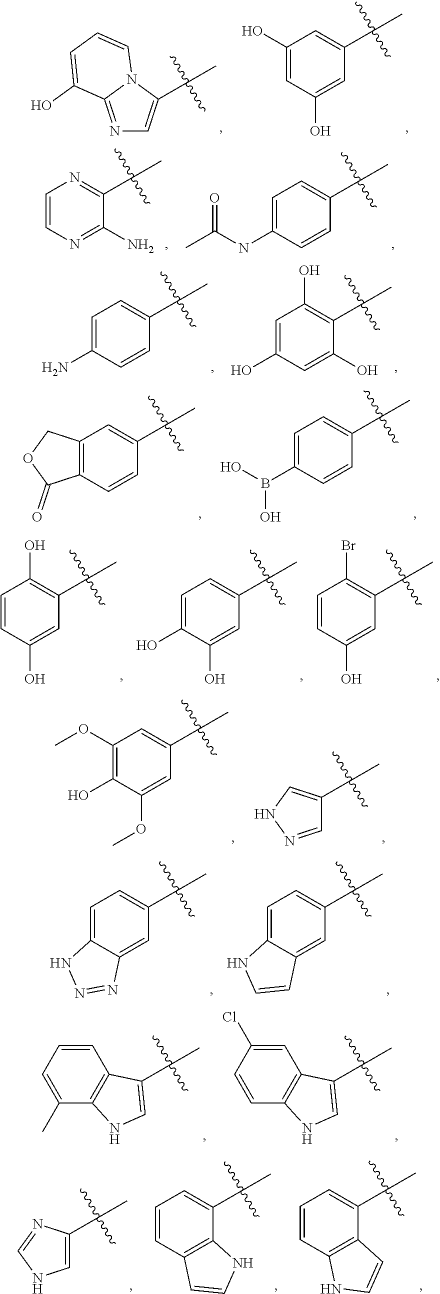

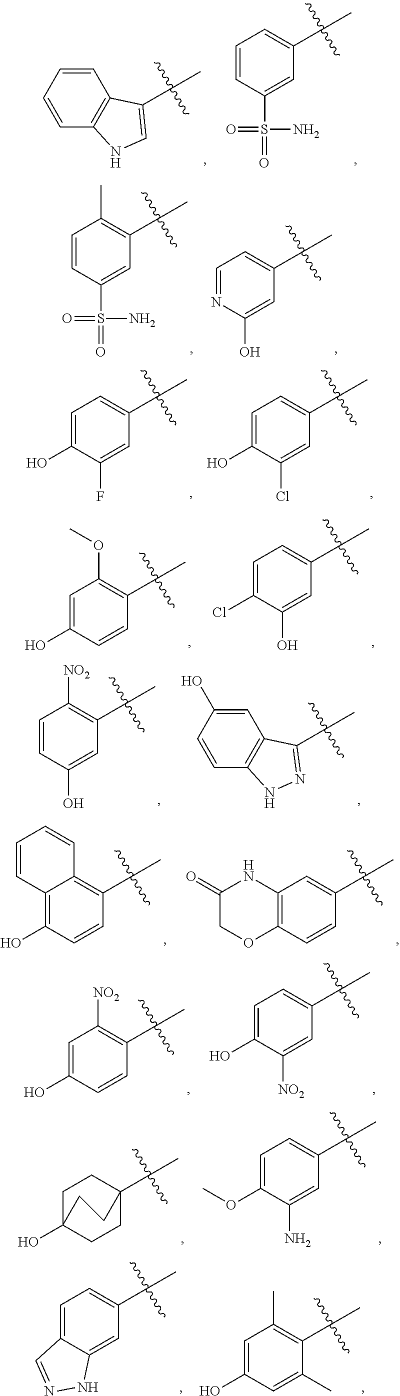

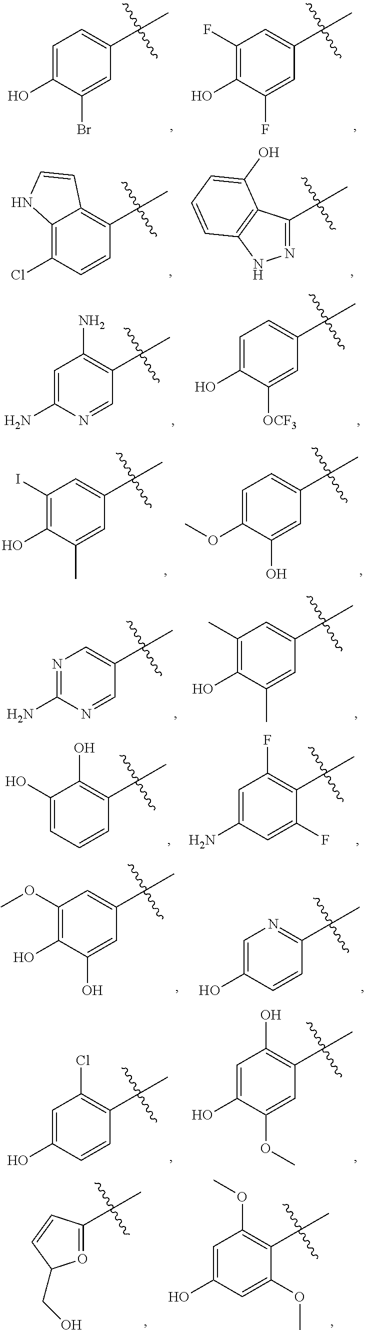

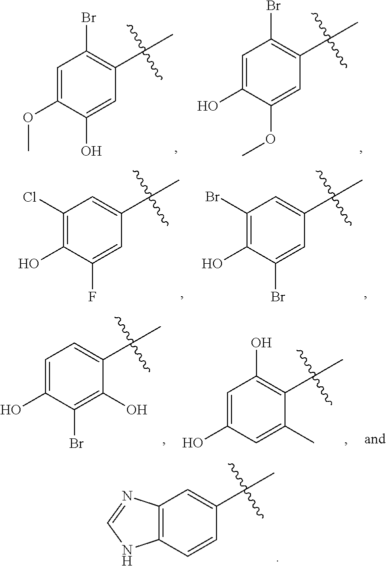

Such embodiments are not limited to a particular definition for R2.

In some embodiments, R2 is selected from hydrogen. CH₃, and

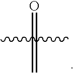 [SMILES: *=O]

Such embodiments are not limited to a particular definition for R3.

In some embodiments, R3 is selected from hydrogen, and CH₃ (e.g., [symbol] , [symbol] ). In some embodiments, R3 is CH₃ (e.g., [symbol] ). In some embodiments, R3 is CH₃ (e.g., [symbol] ).

Such embodiments are not limited to a particular definition for R4.

In some embodiments, R4 is selected from

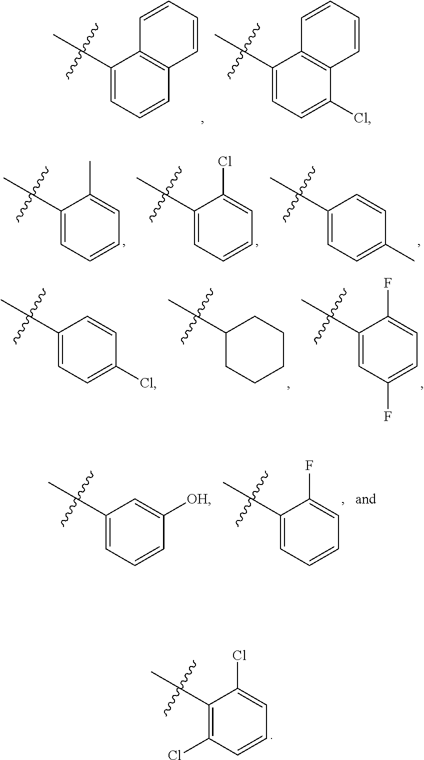

Such embodiments are not limited to a particular definition for R5.

In some embodiments, R5 is selected from hydrogen, CH₃ (e.g., [symbol] , [symbol] ), and

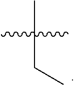 [SMILES: *CC]

Such embodiments are not limited to a particular definition for R6.

In some embodiments, R6 is selected from hydrogen, CH₃,

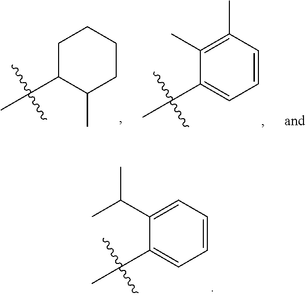

In certain embodiments, compounds encompassed within Formula IV are provided:

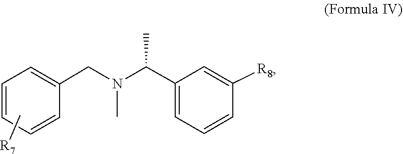

wherein R7 can be monosubstituted or disubstituted with one being CH or NH2, and the benzene ring can be a heterocyle (e.g., pyridine, pyrimidine); and wherein R8 can be any alkyl or aromatic substitutions.

In some embodiments, the compound is recited in Table 1 (see, Example I) or FIG. 1.

The invention further provides processes for preparing any of the compounds of the present invention.

In certain embodiments, the present invention provides methods for administering a pharmaceutical composition comprising one or more compounds of the present invention to a subject (e.g., a human subject) (e.g., a human subject suffering from or at risk of suffering from a condition related to SARS-CoV-2 infection (e.g., COVID-19)) for purposes of treating, preventing and/or ameliorating the symptoms of a viral infection (e.g., SARS-CoV-2 infection (e.g., COVID-19)).

In such embodiments, the methods are not limited treating, preventing and/or ameliorating the symptoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virus having PL^{pro} protease activity and/or expression.

In such embodiments, administration of the pharmaceutical composition results in suppression of PL^{pro} protease activity within the subject. In some embodiments, administration of the pharmaceutical composition results in suppression of any pathway related activity related to PL^{pro} protease activity within the subject.

In some embodiments, the pharmaceutical composition comprising one or more compounds of the present invention is co-administered with one or more of hydroxychloroquine, dexamethasone, and remdesivir.

In certain embodiments, the present invention provides methods for treating, ameliorating and/or preventing a condition related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition comprising one or more compounds of the present invention. In some embodiments, the pharmaceutical composition comprising one or more compounds of the present invention is configured for oral administration. In some embodiments, the subject is a human subject.

In certain embodiments, the present invention provides methods for treating, ameliorating and/or preventing symptoms related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include HI antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisturization by nebulization, anti-tussives, respiratory stimulants, surgery and alpha 1 antitrypsin.

In certain embodiments, the present invention provides methods for inhibiting viral entry in a cell, comprising exposing the cell to a pharmaceutical composition comprising one or more compounds of the present invention. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising one or more compounds of the present invention results in suppression of PL^{pro} activity within the cell.

In certain embodiments, the present invention provides kits comprising a pharmaceutical composition comprising one or more compounds of the present invention, and one or more of (1) a container, pack, or dispenser, (2) one or more additional agents selected from hydroxychloroquine, dexamethasone, and remdesivir, and (3) instructions for administration.

### BRIEF DESCRIPTION OF THE DRAWINGS

FIG. 1 shows compounds of the present invention encompassed within Formulas I, II or III.

### DETAILED DESCRIPTION OF THE INVENTION

Experiments conducted during the course of developing embodiments for the present invention developed a FRET-based enzymatic assay for the PL^{pro}, which was used in a high-throughput screening to identified potent PL^{pro} inhibitors. Several novel compounds were identified. Structure-activity relationship studies were performed to further optimize the potency and selectivity index of these hits. This effort led to a number of potent SARS-CoV-2 PL^{pro} inhibitors with a new composition of matter having a N-methyl-1-(naphthalen-1-yl)ethan-1-amine (or similar) structure (e.g., compounds Jun9-53-2, Jun9-44-3, Jun9-87-1, Jun9-87-2, Jun9-87-3, and Jun10-69-2 represent highly potent SARS-CoV-2 PL^{pro} inhibitors).

Accordingly, the present invention relates to a new class of small-molecules having a N-methyl-1-(naphthalen-1-yl)ethan-1-amine (or similar) structure which function as inhibitors of the SARS-CoV-2 papain-like protease (PL^{pro}), which function as therapeutics for the treatment of viral infection characterized with PL^{pro} protease activity and/or expression (e.g., COVID-19), and which function as therapeutics for the treatment of other conditions characterized with PL^{pro} protease activity and/or expression.

In a particular embodiment, compounds encompassed within the following formulas are provided:

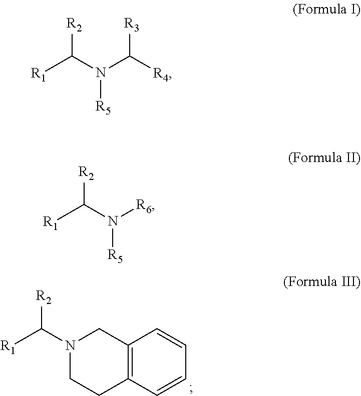

including pharmaceutically acceptable salts, solvates, and/or prodrugs thereof.

Formula I is not limited to a particular chemical moiety for R1, R2, R3, R4, R5, and R6. In some embodiments, the particular chemical moiety for R1, R2, R3, R4, R5, and R6 independently include any chemical moiety that permits the resulting compound to inhibit PLATO protease activity. In some embodiments, the particular chemical moiety R1, R2, R3, R4, R5, and R6 independently include any chemical moiety that permits the resulting compound to prevent viral infection (e.g., COVID-19 infection).

Such embodiments are not limited to a particular definition for R1.

In some embodiments, R1 is selected from hydrogen, methyl,

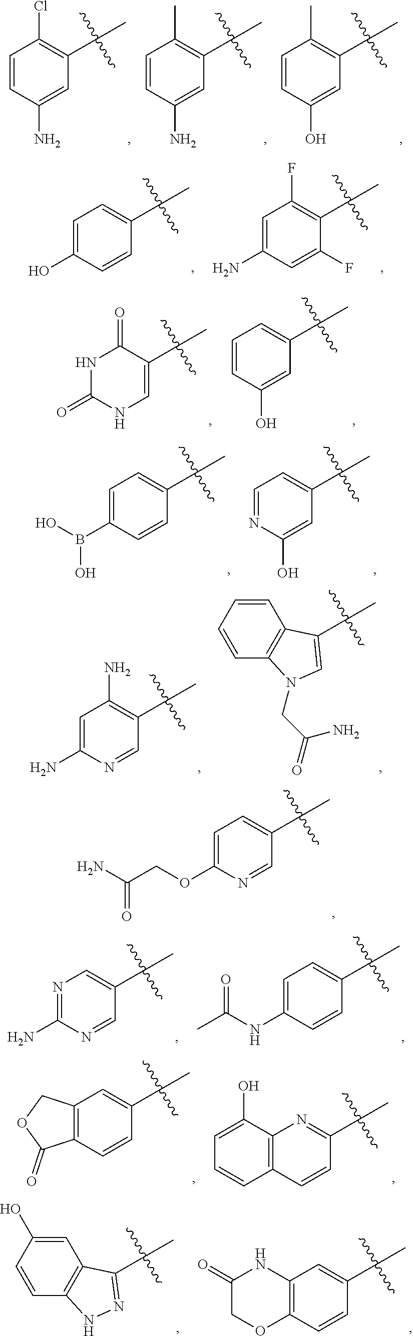

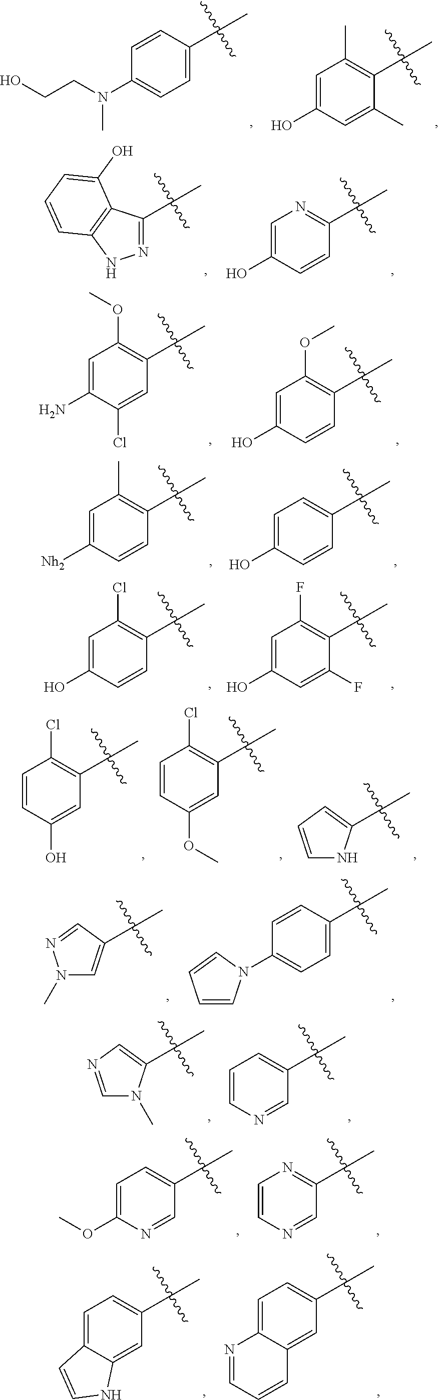

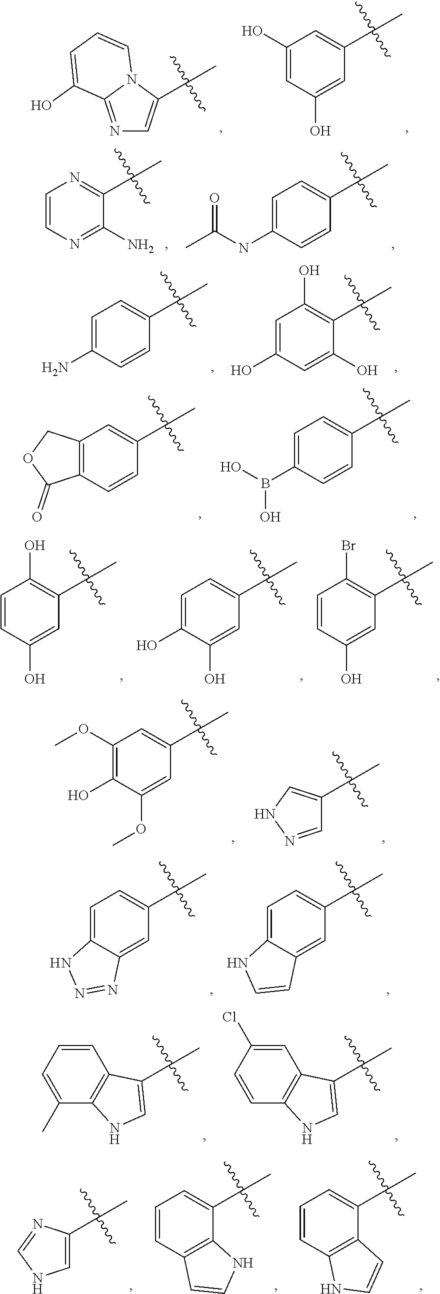

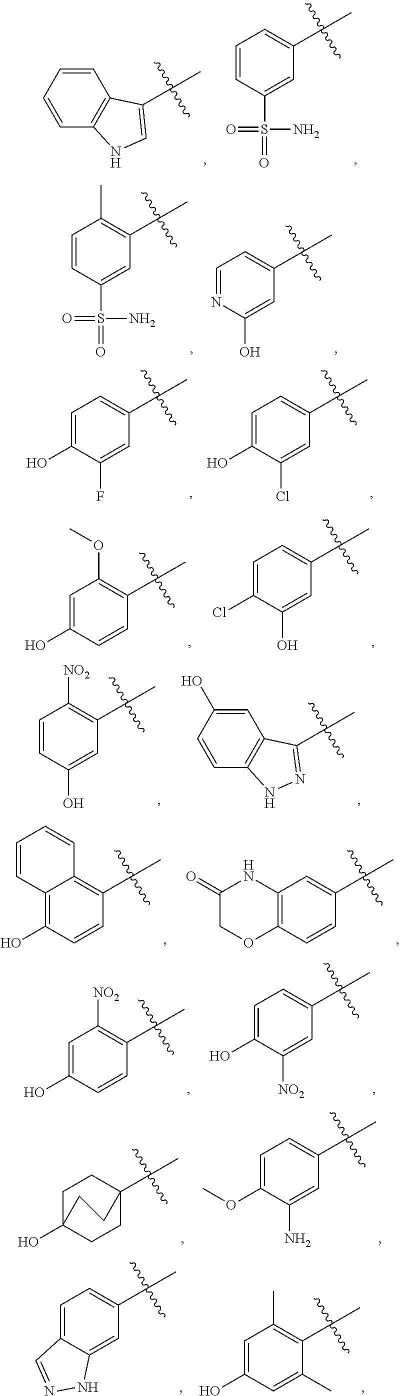

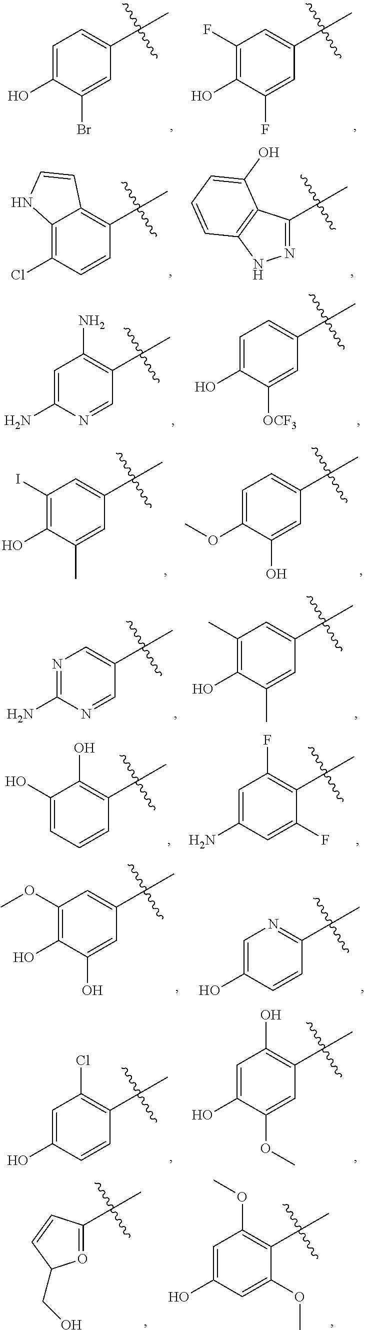

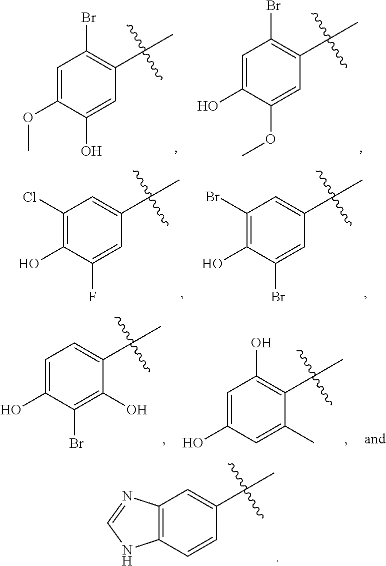

Such embodiments are not limited to a particular definition for R2.

In some embodiments, R2 is selected from hydrogen, CH₃, and

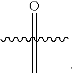 [SMILES: *=O]

Such embodiments are not limited to a particular definition for R3.

In some embodiments, R3 is selected from hydrogen, and CH₃ (e.g., [symbol] , [symbol] ). In some embodiments, R3 is CH₃ (e.g., [symbol] ). In some embodiments, R3 is CH₃ (e.g., [symbol] ).

Such embodiments are not limited to a particular definition for R4.

In some embodiments, R4 is selected from

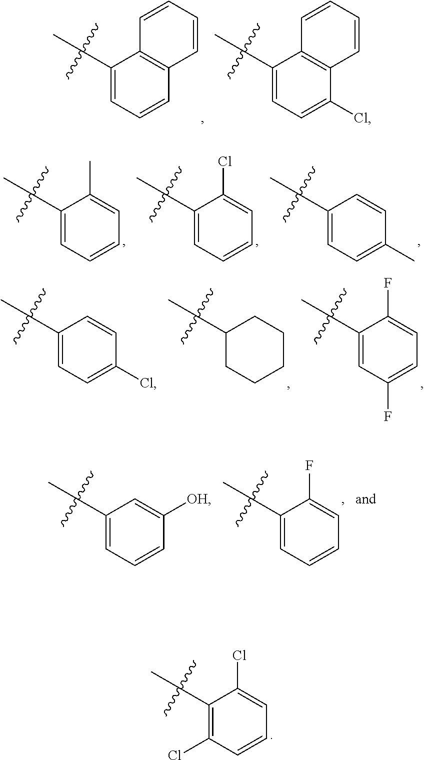

Such embodiments are not limited to a particular definition for R5.

In some embodiments, R5 is selected from hydrogen, CH₃, and

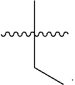 [SMILES: *CC]

Such embodiments are not limited to a particular definition for R6.

In some embodiments, R6 is selected from hydrogen, CH₃,

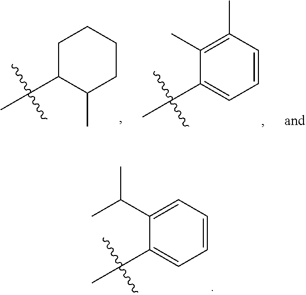

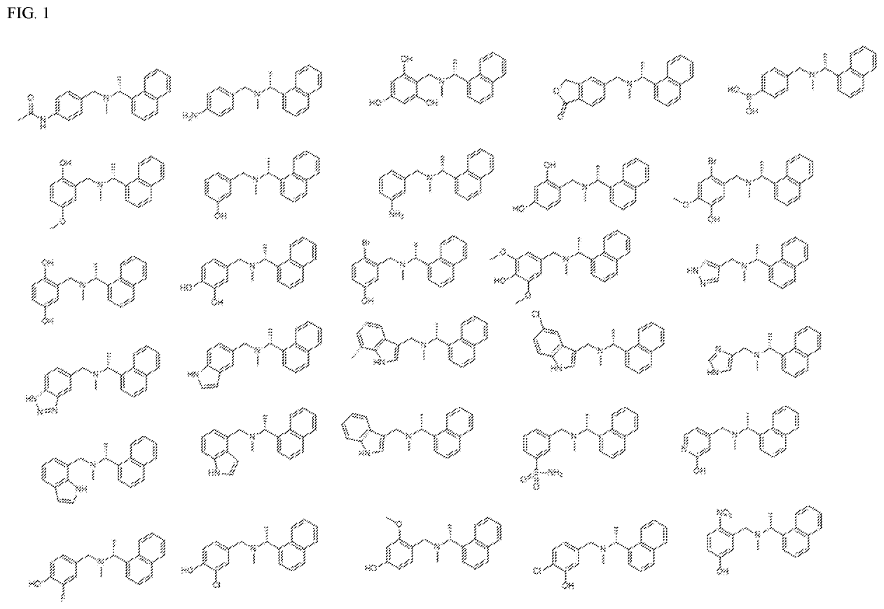

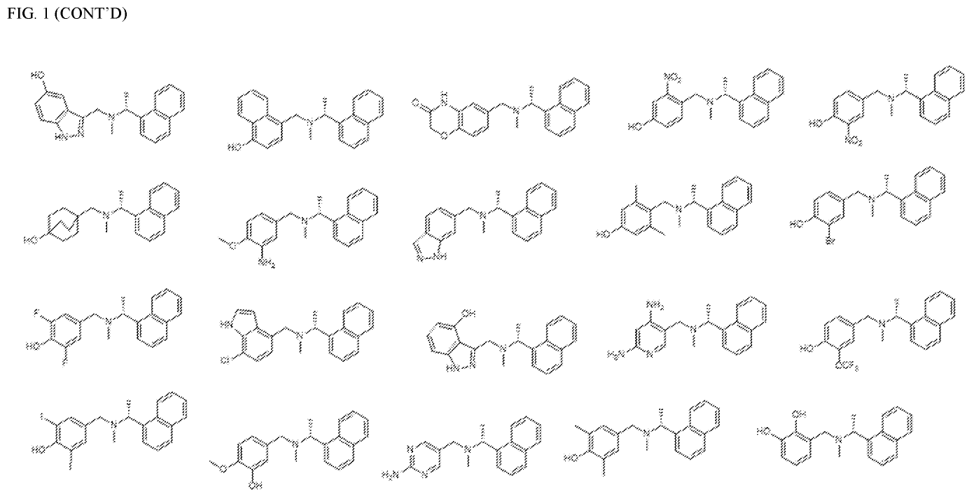

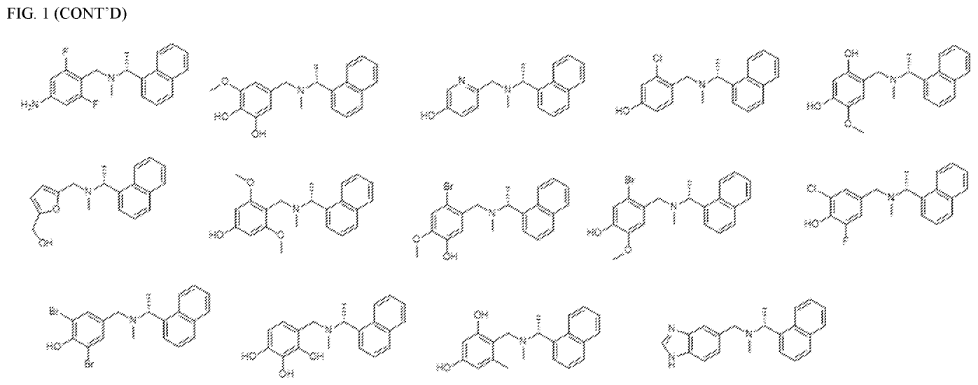

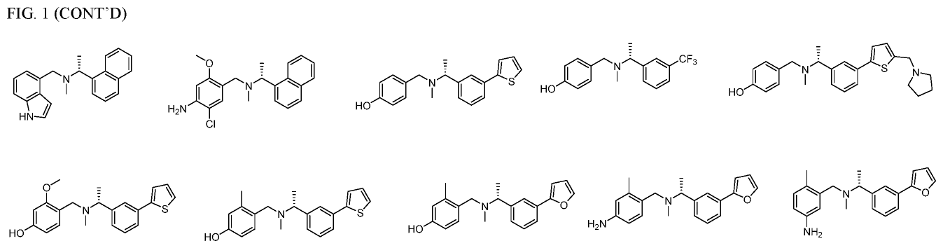

In some embodiments, the compound is recited in Table 1 (see, Example I) or FIG. 1.

An important aspect of the present invention is that the pharmaceutical compositions comprising one or more of compounds of the present invention are useful in treating viral infection (e.g., SARS-CoV-2 infection) and symptoms related to such a viral infection (e.g., fever, fatigue, dry cough, myalgias, dyspnea, acute respiratory distress syndrome, and pneumonia).

Some embodiments of the present invention provide methods for administering an effective amount of a pharmaceutical composition comprising one or more compounds of the present invention and at least one additional therapeutic agent (including, but not limited to, any pharmaceutical agent useful in treating SARS-CoV-2 infection and/or symptoms related to such a viral infection (e.g., fever, fatigue, dry cough, myalgias, dyspnea, acute respiratory distress syndrome, and pneumonia). In some embodiments, the additional agent is one or more of hydroxychloroquine, dexamethasone, and remdesivir.

In certain embodiments, the present invention provides methods for administering a pharmaceutical composition comprising one or more compounds of the present invention to a subject (e.g., a human subject) (e.g., a human subject suffering from or at risk of suffering from a condition related to SARS-CoV-2 infection (e.g., COVID-19)) for purposes of treating, preventing and/or ameliorating the symptoms of a viral infection (e.g., SARS-CoV-2 infection (e.g., COVID-19)).

In such embodiments, the methods are not limited treating, preventing and/or ameliorating the symptoms of a particular type or kind of viral infection. In some embodiments, the viral infection is a SARS-CoV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and West Nile virus. In some embodiments, the viral infection is associated with any virus having PL^{pro} protease activity and/or expression.

In such embodiments, administration of the pharmaceutical composition results in suppression of PL^{pro} protease activity within the subject. In some embodiments, administration of the pharmaceutical composition results in suppression of any pathway related activity related to PL^{pro} protease activity within the subject.

In some embodiments, the pharmaceutical composition comprising one or more compounds of the present invention is co-administered with one or more of hydroxychloroquine, dexamethasone, and remdesivir.

In certain embodiments, the present invention provides methods for treating, ameliorating and/or preventing a condition related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the viral infection is a SARS-CoV-2 viral infection.

In certain embodiments, the present invention provides methods for treating, ameliorating and/or preventing SARS-CoV-2 infection (e.g., COVID-19) in a subject, comprising administering to the subject a pharmaceutical composition comprising one or more compounds of the present invention. In some embodiments, the pharmaceutical composition comprising one or more compounds of the present invention is configured for oral administration. In some embodiments, the subject is a human subject.

In certain embodiments, the present invention provides methods for treating, ameliorating and/or preventing symptoms related to viral infection in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing symptoms related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the one or more symptoms related to viral infection includes, but is not limited to, fever, fatigue, dry cough, myalgias, dyspnea, acute respiratory distress syndrome, and pneumonia.

In certain embodiments, the present invention provides methods for treating, ameliorating and/or preventing acute respiratory distress syndrome in a subject, comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing acute respiratory distress syndrome related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In certain embodiments, the present invention provides methods for treating, ameliorating and/or preventing pneumonia related to SARS-CoV-2 infection (e.g., COVID-19) in a subject, comprising administering to the subject a pharmaceutical composition comprising one or more compounds of the present invention. In some embodiments, the pharmaceutical composition is configured for any manner of administration (e.g., oral, intravenous, topical). In some embodiments, the subject is a human subject. In some embodiments, the subject is a human subject suffering from or at risk of suffering from a condition related to SARS-CoV-2 infection (e.g., COVID-19). In some embodiments, the subject is a human subject suffering from a SARS-CoV-2 viral infection.

In some embodiments involving the treatment of acute respiratory distress syndrome and/or pneumoina, the pharmaceutical composition is administered in combination with a known agent to treat respiratory diseases. Known or standard agents or therapies that are used to treat respiratory diseases include, anti-asthma agent/therapies, anti-rhinitis agents/therapies, anti-sinusitis agents/therapies, anti-emphysema agents/therapies, anti-bronchitis agents/therapies or anti-chronic obstructive pulmonary disease agents/therapies. Anti-asthma agents/therapies include mast cell degranulation agents, leukotriene inhibitors, corticosteroids, beta-antagonists, IgE binding inhibitors, anti-CD23 antibody, tryptase inhibitors, and VIP agonists. Anti-allergic rhinitis agents/therapies include HI antihistamines, alpha-adrenergic agents, and glucocorticoids. Anti-chronic sinusitis therapies include, but are not limited to surgery, corticosteroids, antibiotics, anti-fungal agents, salt-water nasal washes or sprays, anti-inflammatory agents, decongestants, guaifensesin, potassium iodide, luekotriene inhibitors, mast cell degranulating agents, topical moisterizing agents, hot air inhalation, mechanical breathing devices, enzymatic cleaners and antihistamine sprays. Anti-emphysema, anti-bronchitis or anti-chronic obstructive pulmonary disease agents/therapies include, but are not limited to oxygen, bronchodilator agents, mycolytic agents, steroids, antibiotics, anti-fungals, moisturization by nebulization, anti-tussives, respiratory stimulants, surgery and alpha 1 antitrypsin.

In certain embodiments, the present invention provides methods for inhibiting viral entry in a cell, comprising exposing the cell to a pharmaceutical composition comprising one or more compounds of the present invention. In some embodiments, the cell is at risk of viral infection (e.g., a cell at risk of SARS-CoV-2 infection). In some embodiments, the cell has been exposed to a virus (e.g., a cell currently exposed to SARS-CoV-2). In some embodiments, the cell is in culture. In some embodiments, the cell is a living cell in a subject (e.g., a human subject) (e.g., a human subject suffering from COVID-19) (e.g., a human subject at risk of suffering from COVID-19). In some embodiments, exposure of the cell to the pharmaceutical composition comprising one or more compounds of the present invention results in suppression of PL^{pro} activity within the cell.

In certain embodiments, the present invention provides kits comprising a pharmaceutical composition comprising one or more compounds of the present invention, and one or more of (1) a container, pack, or dispenser, (2) one or more additional agents selected from hydroxychloroquine, dexamethasone, and remdesivir, and (3) instructions for administration.

Compositions within the scope of this invention include all pharmaceutical compositions contained in an amount that is effective to achieve its intended purpose. While individual needs vary, determination of optimal ranges of effective amounts of each component is within the skill of the art. Typically, the pharmaceutical agents which function as inhibitors of PL^{pro} protease activity may be administered to mammals, e.g. humans, orally at a dose of 0.0025 to 50 mg/kg, or an equivalent amount of the pharmaceutically acceptable salt thereof, per day of the body weight of the mammal being treated. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the inhibiting agent. The unit dose may be administered one or more times daily as one or more tablets or capsules each containing from about 0.1 to about 10 mg, conveniently about 0.25 to 50 mg of the agent (e.g., small molecule) or its solvates.

In a topical formulation, a compound of the present invention (e.g., a compound having a methyl-acetamido-propanamide structure) may be present at a concentration of about 0.01 to 100 mg per gram of carrier. In a one embodiment, such a compound is present at a concentration of about 0.07-1.0 mg/ml, for example, about 0.1-0.5 mg/ml, and in one embodiment, about 0.4 mg/ml.

In addition to administering a compound of the present invention (e.g., a compound having a methyl-acetamido-propanamide structure) as a raw chemical, it may be administered as part of a pharmaceutical preparation containing suitable pharmaceutically acceptable carriers comprising excipients and auxiliaries which facilitate processing of the compound into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active mimetic peptide(s), together with the excipient.

The pharmaceutical compositions of the invention may be administered to any patient that may experience the beneficial effects of one or more of compounds of the present invention (e.g., compounds having a methyl-acetamido-propanamide structure). Foremost among such patients are mammals, e.g., humans, although the invention is not intended to be so limited. Other patients include veterinary animals (cows, sheep, pigs, horses, dogs, cats and the like).

The pharmaceutical compositions comprising a compound of the present invention (e.g., a compound having a methyl-acetamido-propanamide structure) may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Other pharmaceutical preparations that can be used orally include push-fit capsules made of gelatin, as well as soft, sealed capsules made of gelatin and a plasticizer such as glycerol or sorbitol. The push-fit capsules can contain the active mimetic peptides in the form of granules that may be mixed with fillers such as lactose, binders such as starches, and/or lubricants such as talc or magnesium stearate and, optionally, stabilizers. In soft capsules, the active mimetic peptides are in one embodiment dissolved or suspended in suitable liquids, such as fatty oils, or liquid paraffin. In addition, stabilizers may be added.

Possible pharmaceutical preparations that can be used rectally include, for example, suppositories, which consist of a combination of one or more of the active mimetic peptides with a suppository base. Suitable suppository bases are, for example, natural or synthetic triglycerides, or paraffin hydrocarbons. In addition, it is also possible to use gelatin rectal capsules that consist of a combination of the active mimetic peptides with a base. Possible base materials include, for example, liquid triglycerides, polyethylene glycols, or paraffin hydrocarbons.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

### Example I

SARS-CoV-2 encodes two cysteine proteases, the main protease (M^{pro}) and the papain-like protease (PL^{pro}). Both proteases are validated antiviral drug targets and play essential roles in viral replication. It has been shown that M^{pro} cleaves the viral polyprotein at more than 11 sites, while PL^{pro} cleaves the viral polyprotein at 3 sites. Experiments conducted during the course of developing embodiments for the present invention resulted in the development of a FRET-based enzymatic assay for the PL^{pro}, which was used in a high-throughput screening to identified potent PL^{pro} inhibitors. Several novel compounds were identified. Structure-activity relationship studies were next performed to further optimize the potency and selectivity index of these hits. This effort led to a number of potent SARS-CoV-2 PL^{pro} inhibitors with a new composition of matter as shown in the Table I. Compounds Jun9-53-2, Jun9-44-3, Jun9-87-1, Jun9-87-2, Jun9-87-3, and Jun10-69-2 represent highly potent SARS-CoV-2 PL^{pro} inhibitors.

TABLE 1
SARS-CoV-2 Papain-like protease inhibitors

| | SARS-CoV-2 | |
|---|---|---|
| | Papain-like | |
| | protease | |
| | inhibition in | Cytotoxicity in Vero E6 |
| Chemical Structure | FRET assay | cells |
| 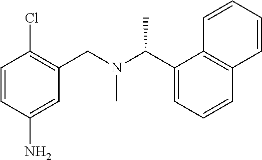 [SMILES: C[C@H](c1cccc2ccccc12)N(C)Cc1cc(N)ccc1Cl] | SARS-CoV-2 PL^{pro} IC₅₀ = 2.71 μM MPro IC₅₀ > 20 μM | Vero E6 CC₅₀ = 32.18 = 1.79 μM |
| Jun9-53-1 | | |

| | | |
|---|---|---|
| 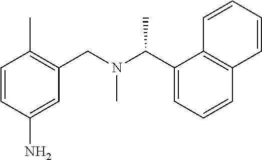 [SMILES: Cc1ccc(N)cc1CN(C)[C@H](C)c1cccc2ccccc12] | SARS-CoV-2 PL^{pro} IC₅₀ = 0.89 μM M^{pro} IC₅₀ > 20 μM | Vero E6 CC₅₀ = 25.70 ± 0.46 μM SARS-CoV-2 antiviral assay in Caco2-hACE2 cells EC₅₀ = 8.89 ± 3.28 μM Vero E6 cells EC₅₀ = 25.19 μM |
| Jun9-53-2 | | |
| 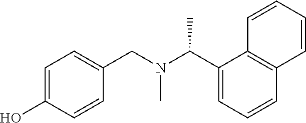 [SMILES: C[C@H](c1cccc2ccccc12)N(C)Cc1ccc(O)cc1] | SARS-CoV-2 PL^{pro} IC₅₀ = 0.76 μM M^{pro} IC₅₀ > 20 μM | Calu-3 CC₅₀ =27.71 ± 1.05 μM SARS-CoV-2 antiviral assay in Caco2-hACE2 cells EC50 = 8.32 ± 1.48 μM Vero E6 cells EC50 = 11.23 ± 6.72 μM |
| Jun9-44-3 | | |
| 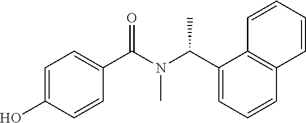 [SMILES: C[C@H](c1cccc2ccccc12)N(C)C(=O)c1ccc(O)cc1] | | |
| 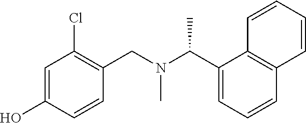 [SMILES: C[C@H](c1cccc2ccccc12)N(C)Cc1ccc(O)cc1Cl] | | |
| 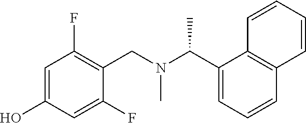 [SMILES: C[C@H](c1cccc2ccccc12)N(C)Cc1c(F)cc(O)cc1F] | | |
| 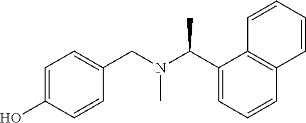 [SMILES: C[C@@H](c1cccc2ccccc12)N(C)Cc1ccc(O)cc1] | | |
| 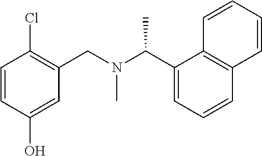 [SMILES: C[C@H](c1cccc2ccccc12)N(C)Cc1cc(O)ccc1Cl] | SARS-CoV-2 PL^{pro} IC₅₀ = 2.66 μM M^{pro} IC₅₀ > 20 μM | Vero E6 CC₅₀ = μM SARS-CoV-2 antiviral assay in Huh7-ACE2 cells EC₅₀ = 5.17 μM |
| Jun9-28-6 | | |
| 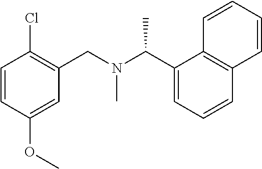 [SMILES: COc1ccc(Cl)c(CN(C)[C@H](C)c2cccc3ccccc23)c1] | SARS-CoV-2 PL^{pro} IC₅₀ > 20 μM | |
| Jun9-26-5 | | |
| 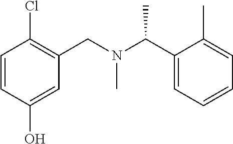 [SMILES: Cc1ccccc1[C@@H](C)N(C)Cc1cc(O)ccc1Cl] | SARS-CoV-2 PL^{pro} IC₅₀ = 4.98 μM | Vero E6 CC₅₀ = 38.76 μM SARS-CoV-2 antiviral assay in Huh7-ACE2 cells EC₅₀ = 3.10 μM |
| Jun9-25-5 (Jun9-13-9) | | |
| Screening hit | | |
| 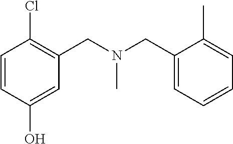 [SMILES: Cc1ccccc1CN(C)Cc1cc(O)ccc1Cl] | SARS-CoV-2 PL^{pro} IC₅₀ > 20 μM | |
| Jun9-26-2 | | |
| 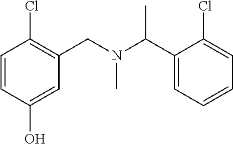 [SMILES: CC(c1ccccc1Cl)N(C)Cc1cc(O)ccc1Cl] | SARS-CoV-2 PL^{pro} IC₅₀ = 7.29 μM | Vero E6 CC₅₀ = 23.27 ^{pro} 1.51 μM |
| Jun9-13-7 | | |
| 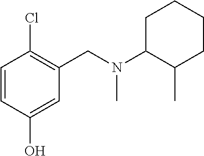 [SMILES: CC1CCCCC1N(C)Cc1cc(O)ccc1Cl] | SARS-CoV-2 PL^{pro} IC₅₀ > 20 μM | |
| Jun9-29-1 | | |
| 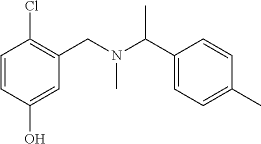 [SMILES: Cc1ccc(C(C)N(C)Cc2cc(O)ccc2Cl)cc1] | SARS-CoV-2 PL^{pro} IC₅₀ = 71.8 μM | |
| Jun9-13-8 | | |
| 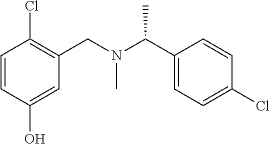 [SMILES: C[C@H](c1ccc(Cl)cc1)N(C)Cc1cc(O)ccc1Cl] | SARS-CoV-2 PL^{pro} IC₅₀ > 20 μM | |
| Jun9-13-4 | | |
| 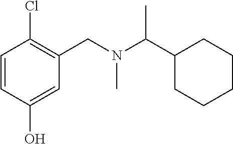 [SMILES: CC(C1CCCCC1)N(C)Cc1cc(O)ccc1Cl] | SARS-CoV-2 PL^{pro} IC₅₀ > 20 μM | |
| Jun9-13-6 | | |
| 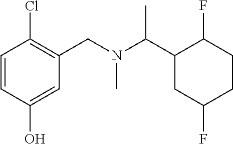 [SMILES: CC(C1CC(F)CCC1F)N(C)Cc1cc(O)ccc1Cl] | SARS-CoV-2 PL^{pro} IC₅₀ > 20 μM | |
| Jun9-29-5 | | |
| 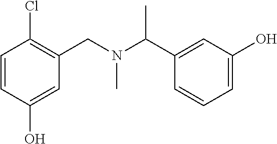 [SMILES: CC(c1cccc(O)c1)N(C)Cc1cc(O)ccc1Cl] | SARS-CoV-2 PL^{pro} IC₅₀ = 26.9 μM | |
| Jun9-29-6 | | |
| 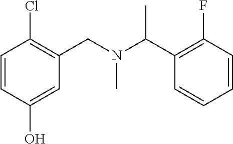 [SMILES: CC(c1ccccc1F)N(C)Cc1cc(O)ccc1Cl] | SARS-CoV-2 PL^{pro} IC₅₀ = 42.9 μM | |
| Jun9-29-7 | | |
| 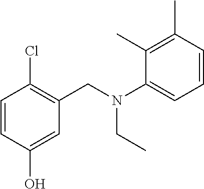 [SMILES: CCN(Cc1cc(O)ccc1Cl)c1cccc(C)c1C] | SARS-CoV-2 PL^{pro} IC₅₀ > 20 μM | |
| Jun9-13-5 | | |
| 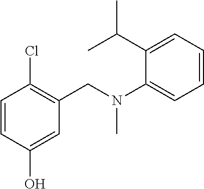 [SMILES: CC(C)c1ccccc1N(C)Cc1cc(O)ccc1Cl] | SARS-CoV-2 PL^{pro} IC₅₀ > 20 μM | |
| Jun9-29-3 | | |
| 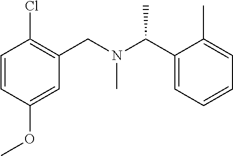 [SMILES: COc1ccc(Cl)c(CN(C)[C@H](C)c2ccccc2C)c1] | SARS-CoV-2 PL^{pro} IC₅₀ = 49.3 μM | Vero E6 CC₅₀ = μM |
| Jun9-25-4 | | |
| 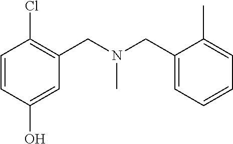 [SMILES: Cc1ccccc1CN(C)Cc1cc(O)ccc1Cl] | SARS-CoV-2 PL^{pro} IC₅₀ > 20 μM | Vero E6 CC₅₀ = μM |
| Jun9-26-2 | | |
| 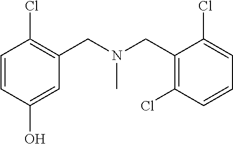 [SMILES: CN(Cc1cc(O)ccc1Cl)Cc1c(Cl)cccc1Cl] | SARS-CoV-2 PL^{pro} IC₅₀ > 20 μM | |
| Jun9-29-4 | | |
| 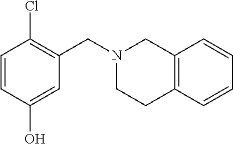 [SMILES: Oc1ccc(Cl)c(CN2CCc3ccccc3C2)c1] | SARS-CoV-2 PL^{pro} IC₅₀ > 20 μM | |
| Jun9-44-5 | | |
| 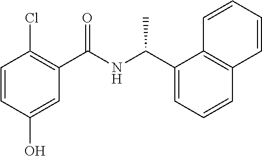 [SMILES: C[C@@H](NC(=O)c1cc(O)ccc1Cl)c1cccc2ccccc12] | SARS-CoV-2 PL^{pro} IC₅₀ = 6.72 μM M^{pro} IC₅₀ > 20 μM | Vero E6 CC₅₀ = 62.08^{pro} + 2.53 μM |
| Jun9-47-2 | | |
| 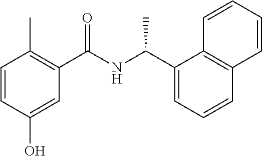 [SMILES: Cc1ccc(O)cc1C(=O)N[C@H](C)c1cccc2ccccc12] | SARS-CoV-2 PL^{pro} IC₅₀ = 2.44 μM M^{pro} IC₅₀ > 20 μM | Vero E6 CC₅₀ = 118.6^{pro} ± 2.14 μM |
| Jun9-47-3 | | |
| 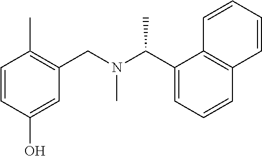 [SMILES: Cc1ccc(O)cc1CN(C)[C@H](C)c1cccc2ccccc12] | SARS-CoV-2 PL^{pro} IC₅₀ = 0.87 μM | |
| Jun9-87-1 | | |
| 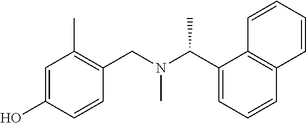 [SMILES: Cc1cc(O)ccc1CN(C)[C@H](C)c1cccc2ccccc12] | SARS-CoV-2 PL^{pro} IC₅₀ = 0.90 μM | |
| Jun9-87-2 | | |
| 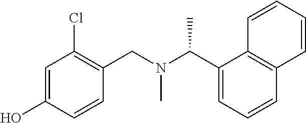 [SMILES: C[C@H](c1cccc2ccccc12)N(C)Cc1ccc(O)cc1Cl] | SARS-CoV-2 PL^{pro} IC₅₀ = 0.80 μM | |
| Jun9-87-3 | | |
| 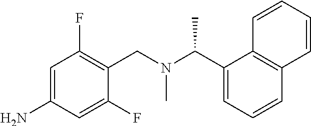 [SMILES: C[C@H](c1cccc2ccccc12)N(C)Cc1c(F)cc(N)cc1F] | SARS-CoV-2 PL^{pro} IC₅₀ = 1.44 μM | |
| Jun9-81-2 | | |
| 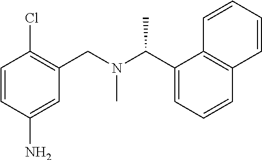 [SMILES: C[C@H](c1cccc2ccccc12)N(C)Cc1cc(N)ccc1Cl] | SARS-CoV-2 PL^{pro} IC₅₀ = 1.85 μM | |
| Jun9-81-3 | | |
| 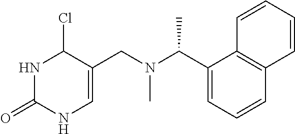 [SMILES: C[C@H](c1cccc2ccccc12)N(C)CC1=CNC(=O)NC1Cl] | SARS-CoV-2 PL^{pro} IC₅₀ = 19.23 μM | |
| Jun9-81-1 | | |
| 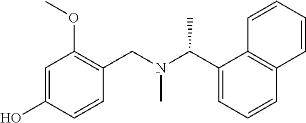 [SMILES: COc1cc(O)ccc1CN(C)[C@H](C)c1cccc2ccccc12] | SARS-CoV-2 PL^{pro} IC₅₀ = 0.56 μM | |
| Jun9-75-5 | | |
| 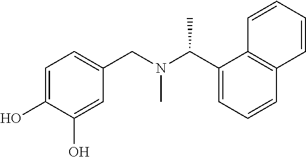 [SMILES: C[C@H](c1cccc2ccccc12)N(C)Cc1ccc(O)c(O)c1] | SARS-CoV-2 PL^{Pro} IC₅₀ = 8.89 μM | |
| Jun9-75-3 | | |
| 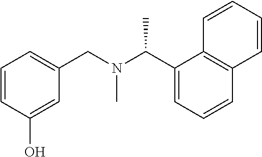 [SMILES: C[C@H](c1cccc2ccccc12)N(C)Cc1cccc(O)c1] | SARS-CoV-2 PL^{pro} IC₅₀ = 1.54 μM | |
| Jun9-75-2 | | |
| 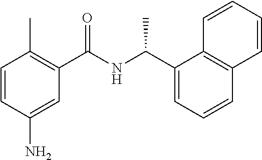 [SMILES: Cc1ccc(N)cc1C(=O)N[C@H](C)c1cccc2ccccc12] | SARS-CoV-2 PL^{pro} IC₅₀ = 1.88 μM M^{pro} IC₅₀ > 20 μM | SARS-CoV-2 antiviral assay in Huh7-ACE2 cells EC₅₀ = 20.2 μM Caco2-ACE2 cells EC₅₀ = 25.14 ± 7.58 μM Vero-E6 cells EC₅₀ = 74.42 μM |
| GRL0617 (Jun8-50-1) | | |
| Reference | | |
| 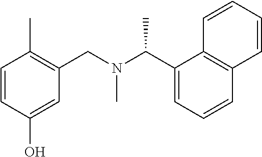 [SMILES: Cc1ccc(O)cc1CN(C)[C@H](C)c1cccc2ccccc12] | SARS-CoV-2 PL^{pro} IC₅₀ = 0.87 μM | |
| Jun9-87-1 | | |
| 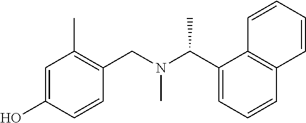 [SMILES: Cc1cc(O)ccc1CN(C)[C@H](C)c1cccc2ccccc12] | SARS-CoV-2 PL^{pro} IC₅₀ = 0.90 μM | |
| Jun9-87-2 | | |
| 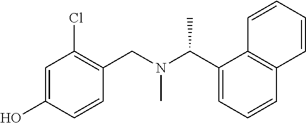 [SMILES: C[C@H](c1cccc2ccccc12)N(C)Cc1ccc(O)cc1Cl] | SARS-CoV-2 PL^{pro} IC₅₀ = 0.80 μM | |
| Jun9-87-3 | | |
| 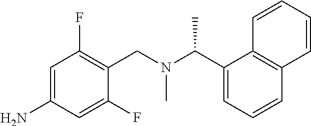 [SMILES: C[C@H](c1cccc2ccccc12)N(C)Cc1c(F)cc(N)cc1F] | SARS-CoV-2 PL^{pro} IC₅₀ = 1.44 μM | |
| Jun9-81-2 | | |
| 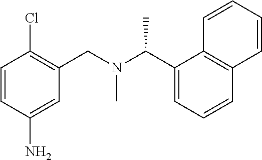 [SMILES: C[C@H](c1cccc2ccccc12)N(C)Cc1cc(N)ccc1Cl] | SARS-CoV-2 PL^{pro} IC₅₀ = 1.85 μM | |
| Jun9-81-3 | | |
| 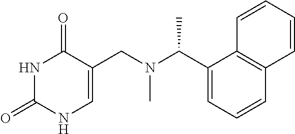 [SMILES: C[C@H](c1cccc2ccccc12)N(C)Cc1c[nH]c(=O)[nH]c1=O] | SARS-CoV-2 PL^{pro} IC₅₀ = 19.23 μM | |
| Jun9-81-1 | | |
| 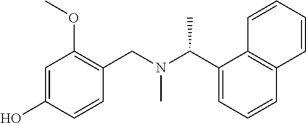 [SMILES: COc1cc(O)ccc1CN(C)[C@H](C)c1cccc2ccccc12] | SARS-CoV-2 PL^{pro} IC₅₀ = 0.56 μM | |
| Jun9-75-5 | | |
| 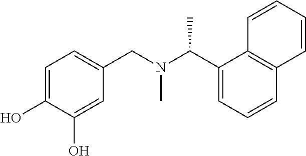 [SMILES: C[C@H](c1cccc2ccccc12)N(C)Cc1ccc(O)c(O)c1] | SARS-CoV-2 PL^{pro} IC₅₀ = 8.89 μM | |
| Jun9-75-3 | | |
| 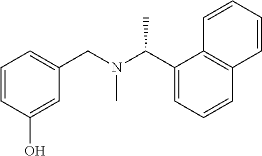 [SMILES: C[C@H](c1cccc2ccccc12)N(C)Cc1cccc(O)c1] | SARS-CoV-2 PL^{pro} IC₅₀ = 1.54 μM | |
| Jun9-75-2 | | |
| 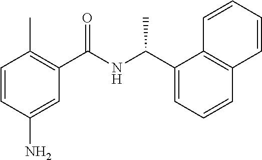 [SMILES: Cc1ccc(N)cc1C(=O)N[C@H](C)c1cccc2ccccc12] | SARS-CoV-2 PL^{pro} IC₅₀ = 1.88 μM M^{pro} IC₅₀ > 20 μM | SARS-CoV-2 antiviral assay in Huh7-ACE2 cells EC₅₀ = 20.2 μM Caco2-ACE2 cells EC₅₀ = 25.14 ± 7.58 μM Vero-E6 cells EC₅₀ = 74.42 μM |
| GRL0617 (Jun8-50-1) | | |
| Reference | | |
| 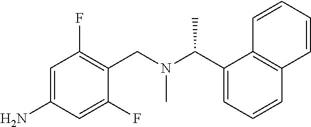 [SMILES: C[C@H](c1cccc2ccccc12)N(C)Cc1c(F)cc(N)cc1F] | SARS-CoV-2 PL^{pro} IC₅₀ = 1.44 μM | |
| Jun9-81-2 | | |
| 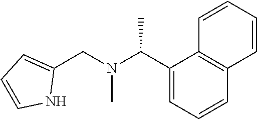 [SMILES: C[C@H](c1cccc2ccccc12)N(C)Cc1ccc[nH]1] | SARS-CoV-2 PL^{pro} IC₅₀ = 7.10 μM | |
| ZX-136-1 | | |
| 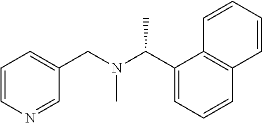 [SMILES: C[C@H](c1cccc2ccccc12)N(C)Cc1cccnc1] | SARS-CoV-2 PL^{pro} IC₅₀ = 9.29 μM | |
| ZX-136-2 | | |
| 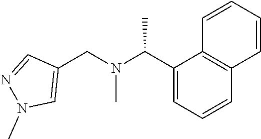 [SMILES: C[C@H](c1cccc2ccccc12)N(C)Cc1cnn(C)c1] | SARS-CoV-2 PL^{pro} IC₅₀ = 10.11 μM | |
| ZX-136-3 | | |
| 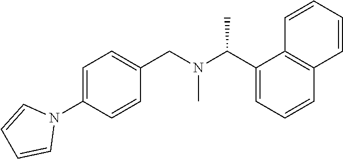 [SMILES: C[C@H](c1cccc2ccccc12)N(C)Cc1ccc(-n2cccc2)cc1] | SARS-CoV-2 PL^{pro} IC₅₀ > 20 μM | |
| ZX-136-4 | | |
| 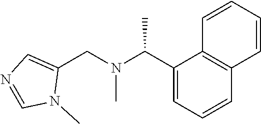 [SMILES: C[C@H](c1cccc2ccccc12)N(C)Cc1cncn1C] | SARS-CoV-2 PL^{pro} IC₅₀ = 8.32 μM | |
| ZX-136-5 | | |
| 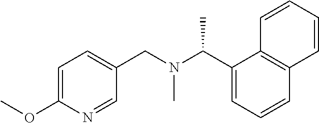 [SMILES: COc1ccc(CN(C)[C@H](C)c2cccc3ccccc23)cn1] | SARS-CoV-2 PL^{pro} IC₅₀ = 11.71 μM | |
| ZX-136-6 | | |
| 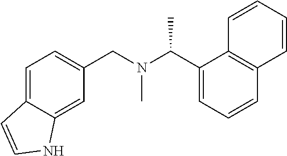 [SMILES: C[C@H](c1cccc2ccccc12)N(C)Cc1ccc2cc[nH]c2c1] | SARS-CoV-2 PL^{pro} IC₅₀ = 1.30 μM | |
| ZX-136-7 | | |
| 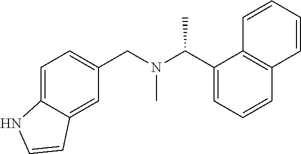 [SMILES: C[C@H](c1cccc2ccccc12)N(C)Cc1ccc2[nH]ccc2c1] | SARS-CoV-2 PL^{pro} IC₅₀ = 1.58 μM | |
| Jun9-80-4 | | |
| 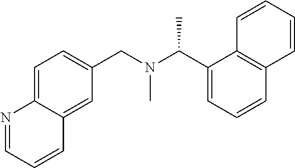 [SMILES: C[C@H](c1cccc2ccccc12)N(C)Cc1ccc2ncccc2c1] | SARS-CoV-2 PL^{pro} IC₅₀ = 5.13 μM | |
| ZX-136-8 | | |
| 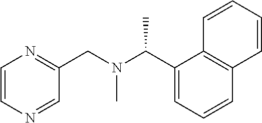 [SMILES: C[C@H](c1cccc2ccccc12)N(C)Cc1cnccn1] | SARS-CoV-2 PL^{pro} IC₅₀ > 20 μM | |
| ZX-136-9 | | |
| 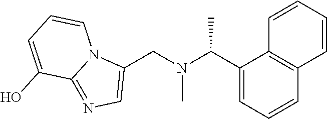 [SMILES: C[C@H](c1cccc2ccccc12)N(C)Cc1cnc2c(O)cccn12] | SARS-CoV-2 PL^{pro} IC₅₀ = 14.35 μM | |
| ZX-136-10 | | |
| 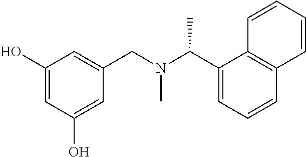 [SMILES: C[C@H](c1cccc2ccccc12)N(C)Cc1cc(O)cc(O)c1] | SARS-CoV-2 PL^{pro} IC₅₀ = 2.72 μM | |
| ZX-128 | | |
| 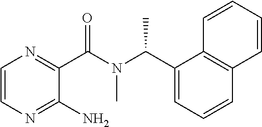 [SMILES: C[C@H](c1cccc2ccccc12)N(C)C(=O)c1nccnc1N] | SARS-CoV-2 PL^{pro} IC₅₀ > 20 μM M^{pro} IC₅₀ > 20 μM | |
| ZX-129 | | |
| 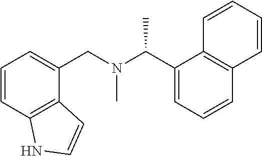 [SMILES: C[C@H](c1cccc2ccccc12)N(C)Cc1cccc2[nH]ccc12] | SARS-CoV-2 PL^{pro} IC₅₀ = 0.62 μM | |
| Jun9-75-4 | | |
| 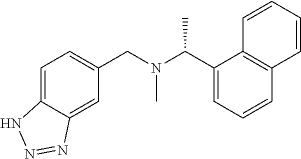 [SMILES: C[C@H](c1cccc2ccccc12)N(C)Cc1ccc2[nH]nnc2c1] | SARS-CoV-2 PL^{pro} IC₅₀ = 2.47 μM | |
| Jun9-84-2 | | |
| 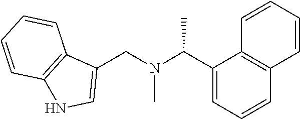 [SMILES: C[C@H](c1cccc2ccccc12)N(C)Cc1c[nH]c2ccccc12] | SARS-CoV-2 PL^{pro} IC₅₀ = 0.67 μM | |
| Jun9-84-3 | | |
| 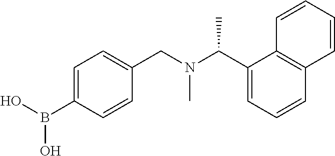 [SMILES: C[C@H](c1cccc2ccccc12)N(C)Cc1ccc(B(O)O)cc1] | SARS-CoV-2 PL^{pro} IC₅₀ = 1.30 μM | |
| Jun9-84-4 | | |
| 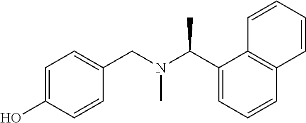 [SMILES: C[C@@H](c1cccc2ccccc12)N(C)Cc1ccc(O)cc1] | SARS-CoV-2 PL^{pro} IC₅₀ > 20 μM | |
| Jun9-84-5 | | |
| 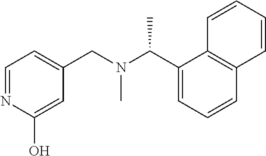 [SMILES: C[C@H](c1cccc2ccccc12)N(C)Cc1ccnc(O)c1] | SARS-CoV-2 PL^{pro} IC₅₀ > 20 μM | |
| Jun9-84-6 | | |
| 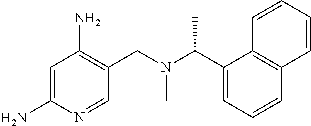 [SMILES: C[C@H](c1cccc2ccccc12)N(C)Cc1cnc(N)cc1N] | SARS-CoV-2 PL^{pro} IC₅₀ = 5.62 μM | |
| Jun9-84-7 | | |
| 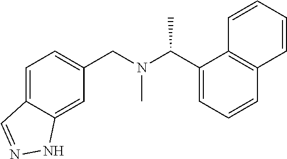 [SMILES: C[C@H](c1cccc2ccccc12)N(C)Cc1ccc2cn[nH]c2c1] | SARS-CoV-2 PL^{pro} IC₅₀ = 0.66 μM | |
| Jun9-85-1 | | |
| 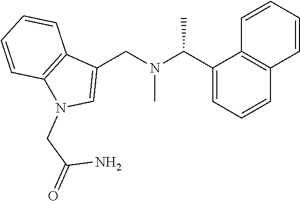 [SMILES: C[C@H](c1cccc2ccccc12)N(C)Cc1cn(CC(N)=O)c2ccccc12] | SARS-CoV-2 PL^{pro} IC₅₀ = 1.15 μM | |
| Jun9-85-2 | | |
| 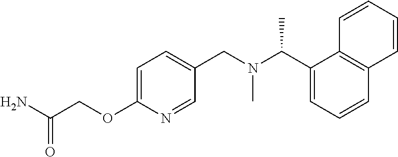 [SMILES: C[C@H](c1cccc2ccccc12)N(C)Cc1ccc(OCC(N)=O)nc1] | SARS-CoV-2 PL^{pro} IC₅₀ = 2.84 μM | |
| Jun9-85-4 | | |
| 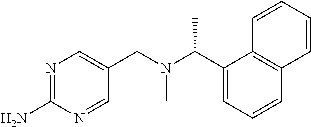 [SMILES: C[C@H](c1cccc2ccccc12)N(C)Cc1cnc(N)nc1] | SARS-CoV-2 PL^{pro} IC₅₀ = 16.59 μM | |
| Jun9-85-5 | | |
| 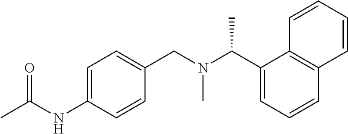 [SMILES: CC(=O)Nc1ccc(CN(C)[C@H](C)c2cccc3ccccc23)cc1] | SARS-CoV-2 PL^{pro} IC₅₀ = 4.42 μM | |
| Jun9-85-6 | | |
| 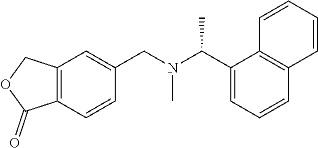 [SMILES: C[C@H](c1cccc2ccccc12)N(C)Cc1ccc2c(c1)COC2=O] | SARS-CoV-2 PL^{pro} IC₅₀ = 6.41 μM | |
| Jun9-85-7 | | |
| 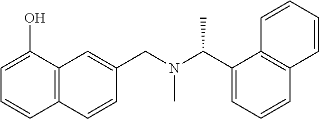 [SMILES: C[C@H](c1cccc2ccccc12)N(C)Cc1ccc2cccc(O)c2c1] | SARS-CoV-2 PL^{pro} IC₅₀ > 20 μM | |
| Jun9-85-8 | | |
| 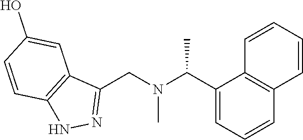 [SMILES: C[C@H](c1cccc2ccccc12)N(C)Cc1n[nH]c2ccc(O)cc12] | SARS-CoV-2 PL^{pro} IC₅₀ = 3.99 μM | |
| Jun9-86-1 | | |
| 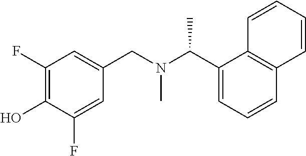 [SMILES: C[C@H](c1cccc2ccccc12)N(C)Cc1cc(F)c(O)c(F)c1] | SARS-CoV-2 PL^{pro} IC₅₀ = 6.49 μM | |
| Jun9-86-2 | | |
| 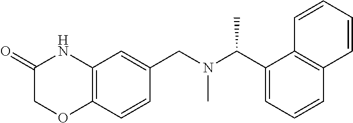 [SMILES: C[C@H](c1cccc2ccccc12)N(C)Cc1ccc2c(c1)NC(=O)CO2] | SARS-CoV-2 PL^{pro} IC₅₀ = 7.01 μM | |
| Jun9-86-3 | | |
| 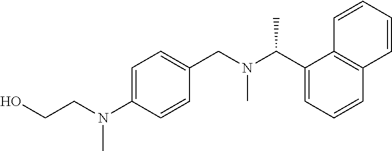 [SMILES: C[C@H](c1cccc2ccccc12)N(C)Cc1ccc(N(C)CCO)cc1] | SARS-CoV-2 PL^{pro} IC₅₀ = 9.22 μM | |
| Jun9-86-4 | | |
| 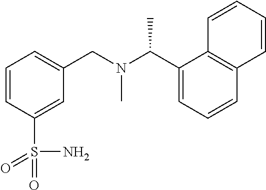 [SMILES: C[C@H](c1cccc2ccccc12)N(C)Cc1cccc(S(N)(=O)=O)c1] | SARS-CoV-2 PL^{pro} IC₅₀ = 6.12 μM | |
| Jun9-86-5 | | |
| 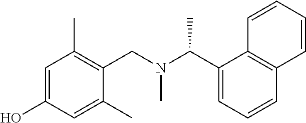 [SMILES: Cc1cc(O)cc(C)c1CN(C)[C@H](C)c1cccc2ccccc12] | SARS-CoV-2 PL^{pro} IC₅₀ = 0.37 μM | |
| Jun9-86-6 | | |
| 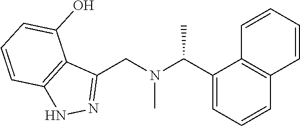 [SMILES: C[C@H](c1cccc2ccccc12)N(C)Cc1n[nH]c2cccc(O)c12] | SARS-CoV-2 PL^{pro} IC₅₀ > 20 μM | |
| Jun9-86-7 | | |
| 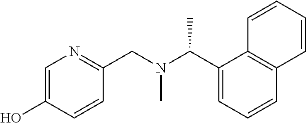 [SMILES: C[C@H](c1cccc2ccccc12)N(C)Cc1ccc(O)cn1] | SARS-CoV-2 PL^{pro} IC₅₀ = 5.50 μM | |
| Jun9-86-8 | | |
| 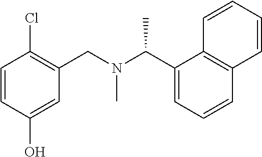 [SMILES: C[C@H](c1cccc2ccccc12)N(C)Cc1cc(O)ccc1Cl] | SARS-CoV-2 PL^{pro} IC₅₀ = 0.87 μM | Vero E6 CC₅₀ = 14.43 ± 0.77 μM |
| Jun9-87-1 | | |
| 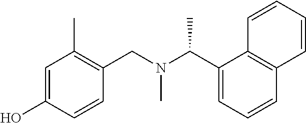 [SMILES: Cc1cc(O)ccc1CN(C)[C@H](C)c1cccc2ccccc12] | SARS-CoV-2 PL^{pro} IC₅₀ = 0.90 μM | Vero E6 CC₅₀ = 60.96 ± 6.79 μM |
| Jun9-87-2 | | |
| 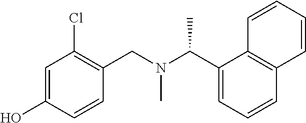 [SMILES: C[C@H](c1cccc2ccccc12)N(C)Cc1ccc(O)cc1Cl] | SARS-CoV-2 PL^{pro} IC₅₀ = 0.80 μM | Vero E6 CC₅₀ = 25.97 ± 2.66 μM |
| Jun9-87-3 | | |
| 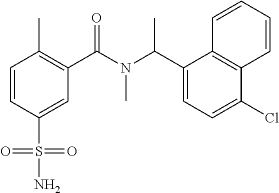 [SMILES: Cc1ccc(S(N)(=O)=O)cc1C(=O)N(C)C(C)c1ccc(Cl)c2ccccc12] | SARS-CoV-2 PL^{pro} IC₅₀ = 3.99 = 0.74 μM | |
| Jun 10-69-2 | | |

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

What is claimed is:

1. A compound encompassed within Formulas I, II or III: 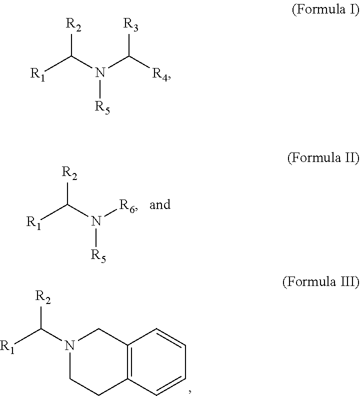
including pharmaceutically acceptable salts, solvates, and/or prodrugs thereof,
wherein each of R1, R2, R3, R4, R5, and R6 independently include any chemical moiety that permits the resulting compound to inhibit PL^{pro} protease activity.

2. The compound of claim 1, wherein each of R1, R2, R3, R4, R5, and R6 independently include any chemical moiety that permits the resulting compound to treat, ameliorate, and/or prevent viral infection (e.g., COVID-19 infection).

3. The compound of claim 1, wherein R1 is selected from hydrogen, methyl,

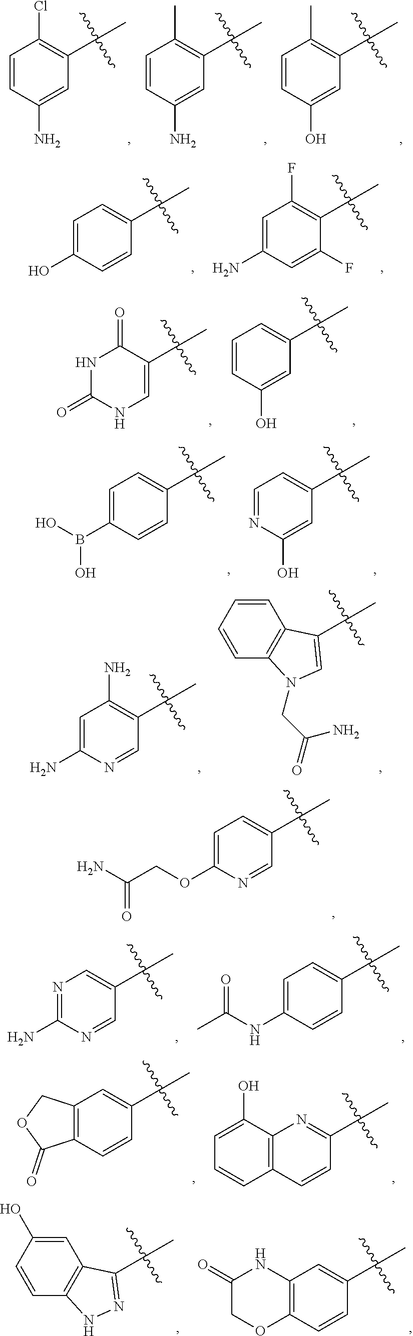

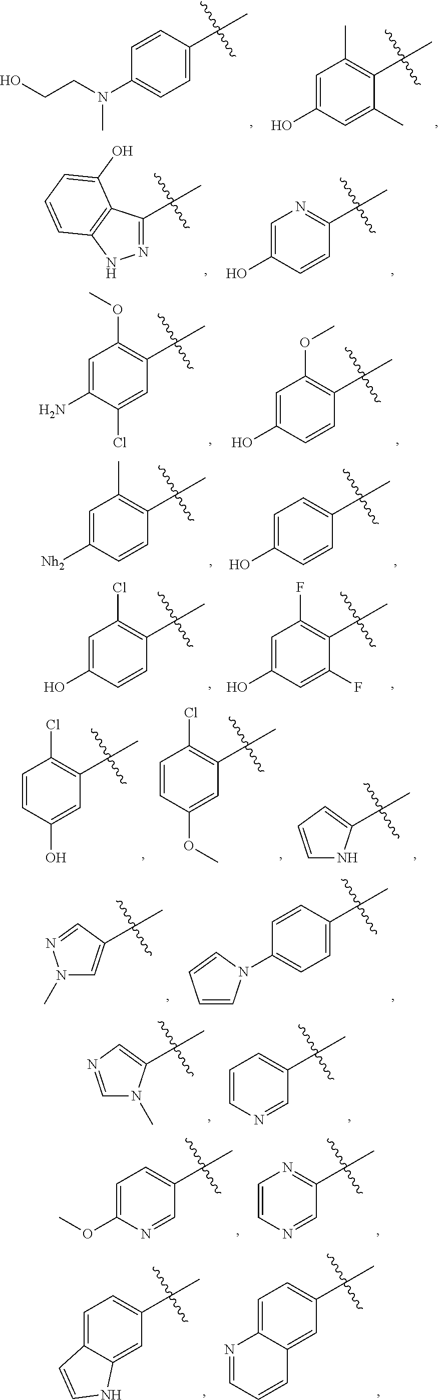

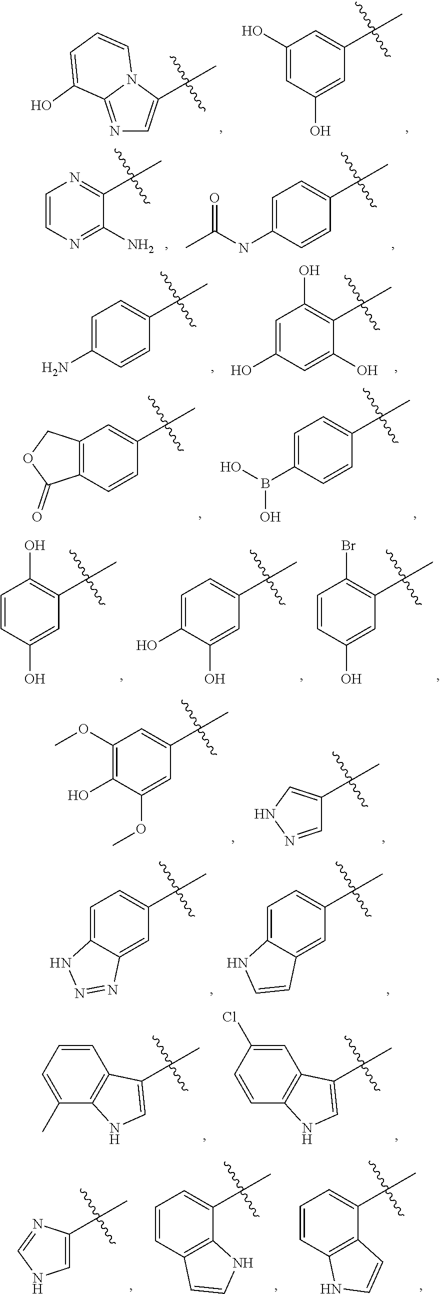

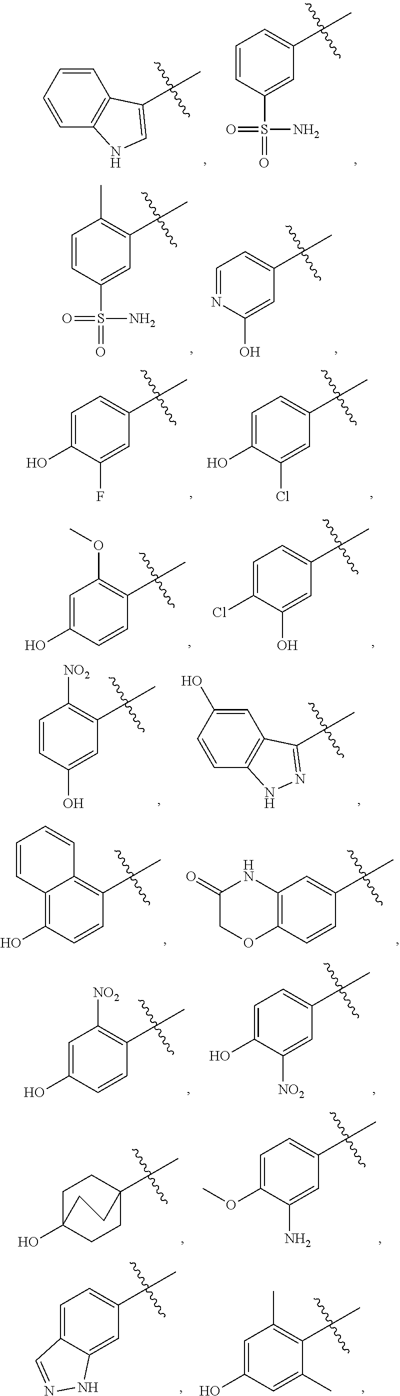

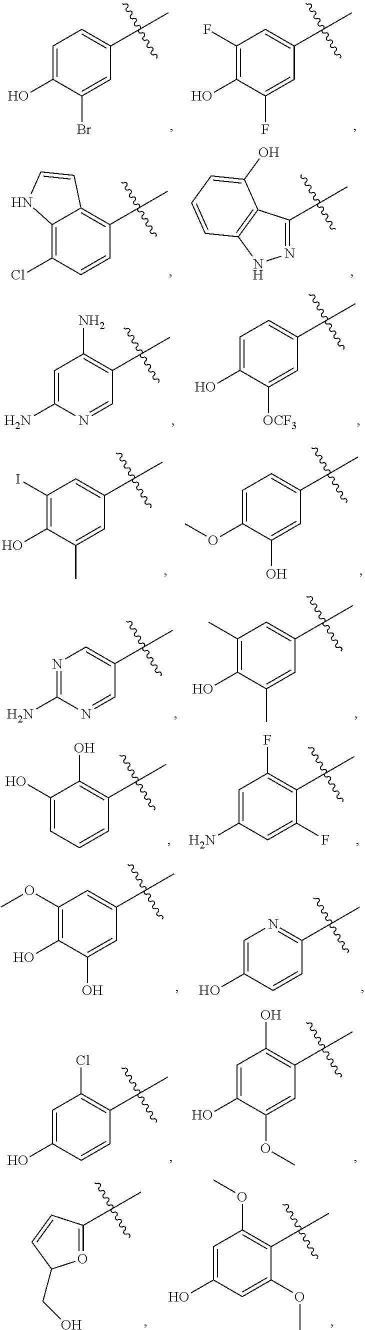

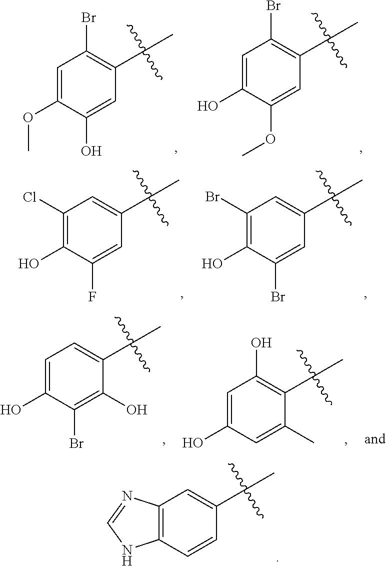

4. The compound of claim 1, wherein R2 is selected from the group consisting of hydrogen, CH₃, and

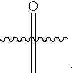 [SMILES: *=O]

5. The compound of claim 1, wherein R3 is selected from the group consisting of hydrogen, and CH₃.

6. The compound of claim 1, wherein R4 is selected from the group consisting of

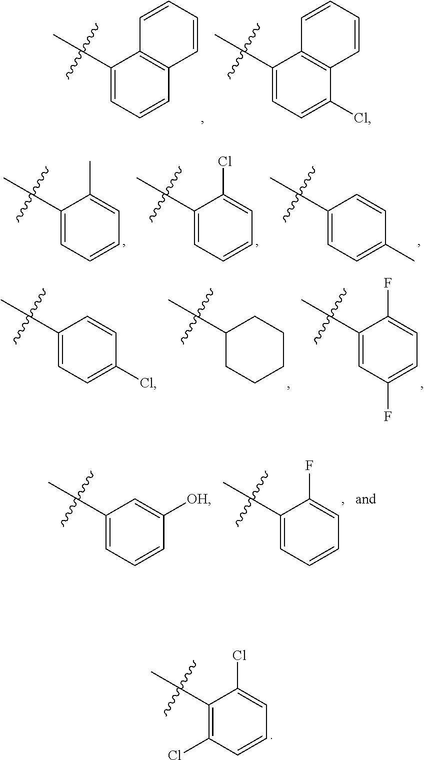

7. The compound of claim 1, wherein R5 is selected from the group consisting of hydrogen, CH₃, and

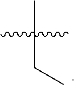 [SMILES: *CC]

8. The compound of claim 1, wherein R6 is selected from the group consisting of hydrogen, CH₃,

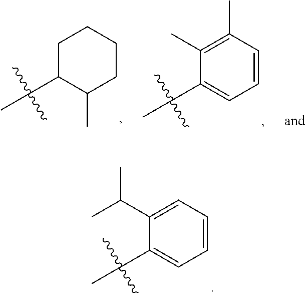

9. The compound of claim 1,
wherein said compound is selected from the group of compounds recited in Table 1 or FIG. 1,
wherein said compound does not include the following compounds:

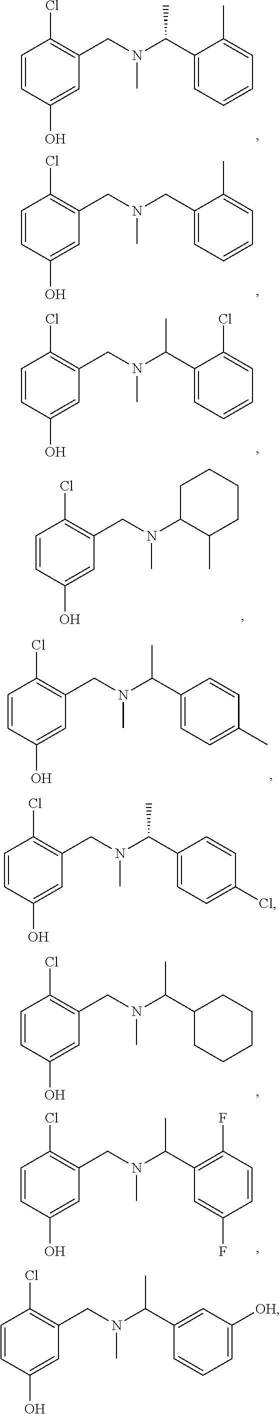

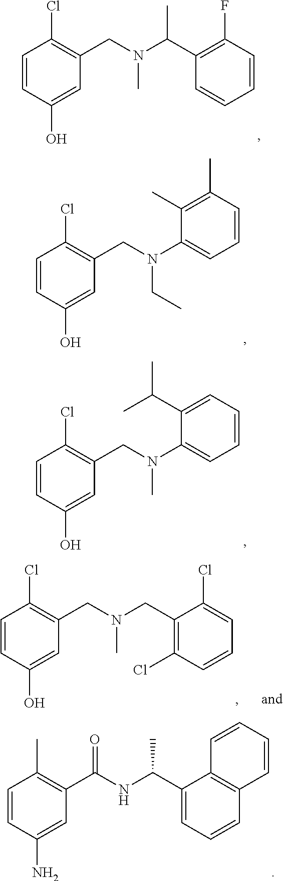

10. A pharmaceutical composition comprising a compound of claim 1.

11. A method for treating, ameliorating and/or preventing a condition related to viral infection in a subject, comprising administering to the subject a therapeutically effective amount of the pharmaceutical composition of claim 10.

12. The method of claim 11, wherein the condition related to viral infection is SARS-CoV-2 infection (e.g., COVID-19).

13. The method of claim 11, wherein the subject is a human subject suffering from or at risk of suffering from a condition related to SARS-CoV-2 infection (e.g., COVID-19).

14. The method of claim 11, wherein the pharmaceutical composition is dispersed in a pharmaceutically acceptable carrier.

15. The method of claim 11, wherein the administering results in suppression of PL^{pro} activity.

16. The method of claim 11, wherein the administering is oral, topical or intravenous.

17. The method of claim 11, further comprising administering to the subject one or more of hydroxychloroquine, dexamethasone, and remdesivir.

18. A method for treating, ameliorating and/or preventing SARS-CoV-2 infection (e.g., COVID-19) in a subject, comprising administering to the subject a therapeutically effective amount of the pharmaceutical composition of claim 10.

19. The method of claim 18, wherein the subject is a human subject suffering from or at risk of suffering from a condition related to SARS-CoV-2 infection (e.g., COVID-19).

20. The method of claim 18, wherein the pharmaceutical composition is dispersed in a pharmaceutically acceptable carrier.

21. The method of claim 18, wherein the administering is oral, topical or intravenous.

22. The method of claim 18, further comprising administering to the subject one or more of hydroxychloroquine, dexamethasone, and remdesivir.

23. The method of claim 18, wherein administration of the pharmaceutical composition results in suppression of PL^{pro} protease activity.

24. A method for treating, ameliorating and/or preventing symptoms related to viral infection in a subject, comprising administering to the subject a therapeutically effective amount of the pharmaceutical composition of claim 10.

25. The method of claim 24, wherein the symptoms related to viral infection in a subject are one or more of fever, fatigue, dry cough, myalgias, dyspnea, acute respiratory distress syndrome, and pneumonia.

26. The method of claim 24, wherein the subject is a human subject suffering from or at risk of suffering from a condition related to SARS-CoV-2 infection (e.g., COVID-19).

27. The method of claim 24, wherein the pharmaceutical composition is dispersed in a pharmaceutically acceptable carrier.

28. The method of claim 24, wherein the administering is oral, intravenous, or topical.

29. The method of claim 24, further comprising administering to the subject one or more of hydroxychloroquine, dexamethasone, and remdesivir.

30. The method of claim 24, wherein administration of the pharmaceutical composition results in suppression of P^{pro} protease activity.

31. A method for treating, ameliorating and/or preventing symptoms related to SARS-CoV-2 infection (e.g., COVID-19) in a subject, comprising administering to the subject a therapeutically effective amount of the pharmaceutical composition of claim 10.

32. The method of claim 31, wherein the symptoms related to viral infection in a subject are one or more of fever, fatigue, dry cough, myalgias, dyspnea, acute respiratory distress syndrome, and pneumonia.

33. The method of claim 31, wherein the pharmaceutical composition is dispersed in a pharmaceutically acceptable carrier.

34. The method of claim 31, wherein the administering is oral, intravenous or topical.

35. The method of claim 31, further comprising administering to the subject one or more of hydroxychloroquine, dexamethasone, and remdesivir.

36. The method of claim 31, wherein administration of the pharmaceutical composition results in suppression of PL^{pro} protease activity.

37. A method for treating, ameliorating and/or preventing acute respiratory distress syndrome in a subject, comprising administering to the subject a therapeutically effective amount of the pharmaceutical composition of claim 10.

38. The method of claim 37, wherein the acute respiratory distress syndrome is related to SARS-CoV-2 infection (e.g., COVID-19).

39. The method of claim 37, wherein the subject is a human subject suffering from or at risk of suffering from a condition related to SARS-CoV-2 infection (e.g., COVID-19).

40. The method of claim 37, wherein the pharmaceutical is dispersed in a pharmaceutically acceptable carrier.

41. The method of claim 37, wherein the administering is oral, intravenous or topical.

42. The method of claim 37, further comprising administering to the subject one or more of hydroxychloroquine, dexamethasone, and remdesivir.

43. The method of claim 37, wherein administration of the pharmaceutical composition results in suppression of PL^{pro} protease activity.

44. A method for treating, ameliorating and/or preventing acute respiratory distress syndrome related to SARS-CoV-2 infection (e.g., COVID-19) in a subject, comprising administering to the subject a therapeutically effective amount of the pharmaceutical composition of claim 10.

45. The method of claim 44, wherein the subject is a human subject suffering from or at risk of suffering from a condition related to SARS-CoV-2 infection (e.g., COVID-19).

46. The method of claim 44, wherein the pharmaceutical composition is dispersed in a pharmaceutically acceptable carrier.

47. The method of claim 44, wherein the administering is oral, intravenous or topical.

48. The method of claim 44, further comprising administering to the subject one or more of hydroxychloroquine, dexamethasone, and remdesivir.

49. The method of claim 44, wherein administration of the pharmaceutical composition results in suppression of PL^{pro} protease activity.

50. A method for treating, ameliorating and/or preventing pneumonia in a subject, comprising administering to the subject a therapeutically effective amount of the pharmaceutical composition of claim 10.

51. The method of claim 50, wherein the pneumonia is related to SARS-CoV-2 infection (e.g., COVID-19).

52. The method of claim 50, wherein the subject is a human subject suffering from or at risk of suffering from a condition related to SARS-CoV-2 infection (e.g., COVID-19).

53. The method of claim 50, wherein the pharmaceutical composition is dispersed in a pharmaceutically acceptable carrier.

54. The method of claim 50, wherein the administering is oral, intravenous or topical.

55. The method of claim 50, further comprising administering an additional agent for treating pneumonia.

56. The method of claim 50, further comprising administering to the subject one or more of hydroxychloroquine, dexamethasone, and remdesivir.

57. The method of claim 50, wherein administration of the pharmaceutical composition results in suppression of PL^{pro} protease activity.

58. A method for treating, ameliorating and/or preventing pneumonia related to SARS-CoV-2 infection (e.g., COVID-19) in a subject, comprising administering to the subject a therapeutically effective amount of the pharmaceutical composition of claim 10.

59. The method of claim 58, wherein the subject is a human subject suffering from or at risk of suffering from a condition related to SARS-CoV-2 infection (e.g., COVID-19).

60. The method of claim 58, wherein the pharmaceutical composition is dispersed in a pharmaceutically acceptable carrier.

61. The method of claim 58, wherein the administering is oral, intravenous or topical.

62. The method of claim 58, further comprising administering to the subject one or more of hydroxychloroquine, dexamethasone, and remdesivir.

63. The method of claim 58, wherein administration of the pharmaceutical composition results in suppression of PL^{pro} protease activity.
